(19) United States
(12) Patent Application Publication
(10) Pub. No.: US 2015/0035943 A1
(43) Pub. Date: Feb. 5, 2015

(54) In-Ear Orthotic for Relieving Temporomandibular Joint-Related Symptoms

(71) Applicant: United Sciences, LLC, Atlanta, GA (US)

(72) Inventors: Karol Constantine Hatzilias, Atlanta, GA (US); Mayoor Patel, Suwanee, GA (US); Wess Eric Sharpe, Atlanta, GA (US); William Jacob Thompson, Cornelia, GA (US)

(73) Assignee: United Sciences, LLC, Atlanta, GA (US)

(21) Appl. No.: 13/958,227

(22) Filed: Aug. 2, 2013

(51) Int. Cl. A61F 2/30; A61B 5/00; A61B 5/0488; H04N 13/02; A61B 5/11

(52) U.S. Cl. CPC A61F 2/3099; H04N 13/0203; A61B 5/11; A61B 5/0488; A61B 5/0059; A61F 2002/183
USPC 348/46; 600/590; 600/546; 600/476; 623/10

(57) ABSTRACT

An in-ear orthotic with one or more features to help manage or reduce pain, discomfort, or other symptoms associated with temporomandibular joint disorder. Also disclosed are methods of using optical scanning to create a three dimensional replication of the ear canal that is used to design a customized in-ear orthotic to help manage one or more symptoms of temporomandibular joint disorder.

### FIELD OF THE INVENTION

Embodiments of the invention generally relate to an in-ear orthotic for managing temporomandibular joint-related symptoms.

### BACKGROUND OF THE INVENTION

The temporomandibular joint (TMJ) includes a small articular disc of cartilage positioned between the mandible (lower jaw) and the temporal bone of the skull. As shown in FIGS. 2-7, the TMJ is the articulation between the two bones, allowing the lower jaw (mandible) to rotate and glide freely in various planes as the jaw opens, closes, protracts, retracts, and moves laterally and medially. The TMJ sits in front of the ear on each side of the head and abuts the ear canal (the external auditory meatus). As shown in FIGS. 2 and 7, the inferior surface of the TMJ disc 22 sits against the condyle 20 of the mandible and the superior surface of the TMJ disc sits against the fossa 24 of the temporal bone.

The TMJ moves whenever a person chews, talks, swallows, yawns, or otherwise moves his jaw and is therefore one of the most frequently moved joints in the body. As shown in FIGS. 3-5, the TMJ both rotates and translates (glides) during movement of the jaw. Specifically, the TMJ is divided into compartments: the inferior compartment, which allows the condyle 20 to rotate when the jaw first begins to open (FIG. 4), and the superior compartment, which hinges and translates (glides) with the condyle 20 as the jaw continues to open (FIG. 5).

Dysfunction of the TMJ is referred to as TMJ disorder or dysfunction (collectively, “TMD”) and can result from the TMJ becoming inflamed, injured, stressed, displaced (subluxed), dislocated, or otherwise damaged. Some people experience popping or clicking when the articular disc in the TMJ is displaced and then snaps back into position as the jaw moves; limited opening or locking of the jaw; tenderness; pain; and/or discomfort. In some cases, when a person clenches or grinds his teeth (bruxism), the condyle 20 compresses the connective tissue of the TMJ, causing inflammation of the connective tissue surrounding the TMJ (such as connective tissue 26 in FIGS. 2 and 6) and pain. In some cases, the clenching/grinding of teeth not only triggers TMJ-related discomfort, but also may contribute to the onset of TMD and to the subsequent deterioration of the joint.

It is estimated that approximately 75% of the population has at least one sign of TMD. Symptoms associated with TMD can be severe and are not always isolated to the joint itself as symptoms of TMD may present in the head, ears, neck, eyes, teeth, and/or jaw. As such, there remains a need for more effective ways to manage TMD and alleviate one or more symptoms caused from it.

### SUMMARY OF THE INVENTION

The terms “invention,” “the invention,” “this invention” and “the present invention” used in this patent are intended to refer broadly to all of the subject matter of this patent and the patent claims below. Statements containing these terms should not be understood to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to the entire specification of this patent, all drawings and each claim.

In certain embodiments, provided is an orthotic for reduction of one or more symptoms associated with temporomandibular joint disorder. In one embodiment, the orthotic is configured for insertion in a subject's ear canal and may be customized based on the configuration of the subject's ear canal. In some embodiments, the orthotic is customized to the particular subject's ear canal by scanning the ear canal, generating a three dimensional image of the scanned ear canal, and modeling the orthotic off of the generated three dimensional image.

According to one embodiment, provided is a custom in-ear orthotic comprising a surface including at least one feature that is customized to a subject's ear canal, wherein the at least one feature provides a sensory indication to the subject when the subject's jaw moves past a predetermined limit in range of motion, and wherein the in-ear orthotic is customized to the subject's ear canal by scanning the subject's ear canal, generating a three-dimensional image of the scanned ear canal and modeling the in-ear orthotic from the generated three dimensional image.

According to another embodiment, provided is a device adapted to be inserted into an ear canal having a bend for treating discomfort in a joint between a mandible and a corresponding temporal bone, the joint having a disc located between the mandible and the temporal bone and associated musculature, the device having a generally cylindrical core with an exterior surface shaped to substantially conform to a contour of the portion of the ear canal which extends approximately between the entrance to the ear canal and the bend, the device, when inserted, adapted reduce discomfort in the joint, wherein the device is customized to the ear canal by scanning the ear canal to determine a geometry of the ear canal, generating a three-dimensional model of the ear canal using the determined geometry of the ear canal, and modeling the device based on the generated three-dimensional model.

According to another embodiment, provided is a system for reducing one or more symptoms associated with temporomandibular joint disorder comprising: (1) a scanner having a scanner body, the body comprising a hand grip, the body having mounted upon it an ear probe, a tracking illumination emitter, a plurality of tracking illumination sensors, and a display screen, the scanner body having mounted within it an image sensor; the ear probe comprising a wide-angle lens optically coupled to the image sensor, a laser light source, a laser optical element, and a source of non-laser video illumination; the plurality of tracking illumination sensors disposed upon the scanner body so as to sense reflections of tracking illumination emitted from the tracking illumination emitter and reflected from tracking targets installed at positions that are fixed relative to an ear canal; the display screen coupled for data communications to the image sensor, the display screen displaying images of the ear canal, the display screen positioned on the scanner body in relation to the ear probe so that, when the ear probe is positioned for scanning, both the display screen and the ear probe are visible to an operator operating the scanner; and the image sensor coupled for data communications to a data processor, with the data processor configured so that it functions by constructing, in dependence upon a sequence of images captured when the scanned ear is illuminated by laser light and tracked positions of the ear probe inferred from reflections of tracking illumination sensed by the tracking illumination sensors, a 3D image of the interior of the ear canal; and (2) a device that is modeled from the constructed 3D image, wherein the device is adapted to be inserted into the ear canal, the ear canal having a bend for treating discomfort in a joint between a mandible and a corresponding temporal bone, the joint having a disc located between the mandible and the temporal bone and associated musculature, the device having a generally cylindrical core with an exterior surface shaped to substantially conform to a contour of the portion of the ear canal which extends approximately between the entrance to the ear canal and the bend, the device, when inserted, adapted to reduce discomfort in the joint.

According to yet another embodiment, provided is a system for reducing one or more symptoms associated with temporomandibular joint disorder in a subject comprising: (1) a scanner comprising: a scanner body, the body comprising a hand grip, the body having mounted upon it an ear probe, a tracking illumination emitter, a plurality of tracking illumination sensors, and a display screen, the scanner body having mounted within it an image sensor; the ear probe comprising a wide-angle lens optically coupled to the image sensor, a laser light source, a laser optical element, and a source of non-laser video illumination; the plurality of tracking illumination sensors disposed upon the scanner body so as to sense reflections of tracking illumination emitted from the tracking illumination emitter and reflected from tracking targets installed at positions that are fixed relative to an ear canal of the subject; the display screen coupled for data communications to the image sensor, the display screen displaying images of the ear canal, the display screen positioned on the scanner body in relation to the ear probe so that, when the ear probe is positioned for scanning, both the display screen and the ear probe are visible to an operator operating the scanner; and the image sensor coupled for data communications to a data processor, with the data processor configured so that it functions by constructing, in dependence upon a sequence of images captured when the scanned ear is illuminated by laser light and tracked positions of the subject's ear probe inferred from reflections of tracking illumination sensed by the tracking illumination sensors, a 3D image of the interior of the ear canal; and (2) a custom in-ear orthotic modeled from the constructed 3D image of the interior of the subject's ear canal such that the custom orthotic substantially conforms to the subject's ear canal, the in-ear orthotic comprising a surface comprising at least one feature that is customized to the subject's ear canal, wherein the at least one feature provides a sensory indication to the subject's ear canal when the a jaw moves past a predetermined limit.

According to a further embodiment, disclosed is an in-ear device that is customized to a subject's ear canal to substantially deform the ear canal to relieve one or more symptoms associated with temporomandibular joint disorder.

### BRIEF DESCRIPTION OF THE DRAWINGS

A full and enabling disclosure including the best mode of practicing the appended claims and directed to one of ordinary skill in the art is set forth more particularly in the remainder of the specification. The specification makes reference to the following appended figures, in which use of like reference numerals in different features is intended to illustrate like or analogous components.

FIG. 1 is a coronal section illustrating the anatomy of the ear.

FIG. 2 is a sagittal section illustrating the TMJ.

FIGS. 3-5 illustrate the movement of the TMJ as the jaw opens.

FIG. 6 is a transverse section showing the positioning of the TMJ relative to the ear canal.

FIG. 7 is another coronal section showing the TMJ.

FIG. 8 is a perspective view of a custom designed TMD in-ear orthotic according to one embodiment.

FIG. 9 is another view of the orthotic of FIG. 8.

FIG. 10 is another view of the orthotic of FIG. 8.

FIG. 11 is a perspective view of an in-ear orthotic according to another embodiment.

FIG. 12 is a perspective view of an in-ear orthotic according to another embodiment.

FIG. 13 is a perspective view of an in-ear orthotic according to another embodiment.

FIG. 14 is a lateral sagittal view of the orthotic of FIG. 13.

FIG. 15 is a lateral sagittal view of an in-ear orthotic according to another embodiment.

FIG. 16 is a sagittal section showing the distribution of the trigeminal nerve.

FIG. 17A is a line drawing of an example scanner.

FIGS. 17B-17E are line drawings of further example scanners.

FIG. 18 is a line drawing of an even further example scanner.

FIGS. 19A and 19B illustrate projections of laser light onto surfaces of a scanned ear.

FIG. 20 is a flow chart illustrating an example method of constructing a 3D image of a scanned ear.

FIG. 21 is a line drawing illustrating additional example features of an ear probe and image sensor of a scanner according to embodiments of the present invention.

FIG. 22 is a line drawing of an example ear probe (106) of a scanner according to embodiments of the invention.

FIGS. 23A and 23B are drawings of an example optical element and a fan of laser light projected from an ear probe having such an optical element.

FIGS. 24A and 24B are line drawings of a further optical element and a resultant ring of laser light projected from an ear probe having such an optical element.

FIG. 25 illustrates a skin target with partial lateral portions of rings of laser light projected thereon.

FIG. 26 illustrates reflected laser light intensity varying in a bell-curve shape with a thickness of a section of projected laser light.

FIG. 27 is an image captured from reflections of laser light reflected from a conical laser reflective optical element.

FIG. 28 is a line drawing schematically illustrating transforming ridge points to points in scanner space.

FIG. 29 is a line drawing illustrating an example three-dimensional image of an ear canal constructed by use of a data processor from a sequence of 2D images.

FIG. 30 is a 3D image of a scanned ear created by use of a scanner and 3D imaging according to embodiments of the present invention.

FIG. 31 is a line drawing of a scanner capable of detecting the force with which the ear probe is pressed against a surface of the scanned ear for use in calculating a compliance value as an aid to a manufacturer in making comfortable and well fitting objects worn in the ear.

FIG. 32 is a further example scanner according to embodiments of the present invention.

FIG. 33 is a line drawing illustrating a method of determining the location and orientation in ear space of the ear drum of a scanned ear according to a method of structure-from-motion.

### DETAILED DESCRIPTION OF THE DRAWINGS

The subject matter of embodiments of the present invention is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

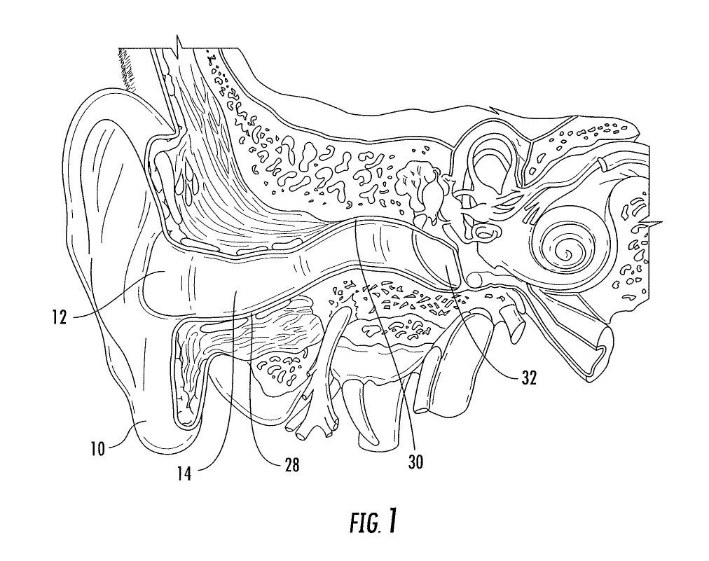

As shown in FIG. 1, the ear canal (auditory canal) 14 extends from the concha 12 and forms a generally-S shaped curve that has constrictions, one at a first bend 28 and another at a second bend 30. These bends help prevent foreign objects from reaching and damaging the ear drum (tympanic membrane) 32. FIG. 1 illustrates the ear canal generally, but much like fingerprints, each person's ear canal is unique.

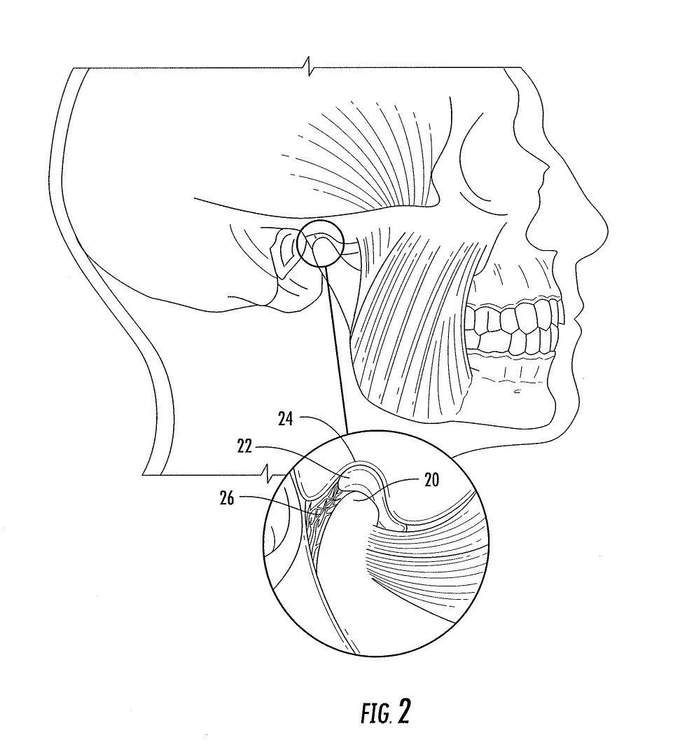

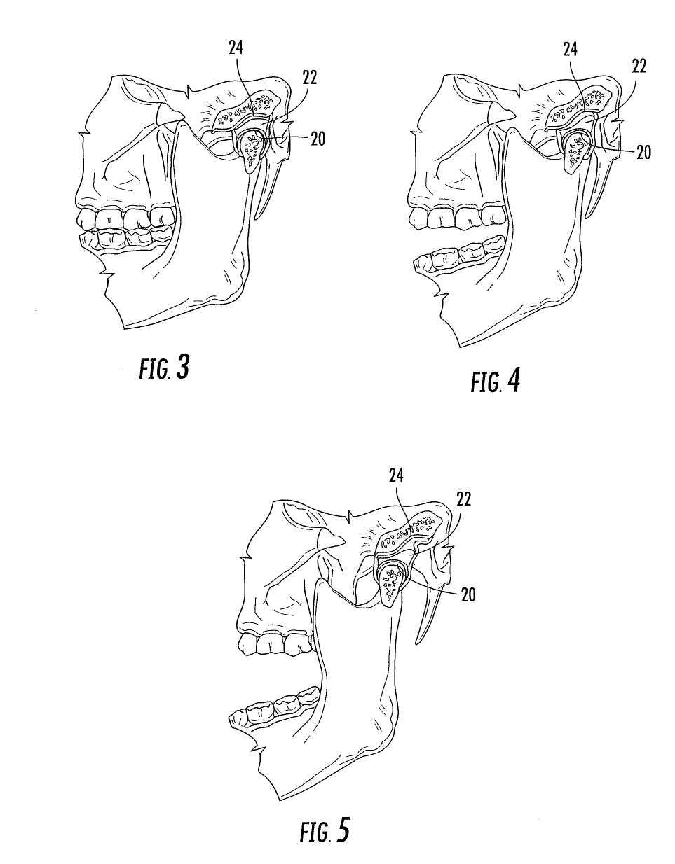

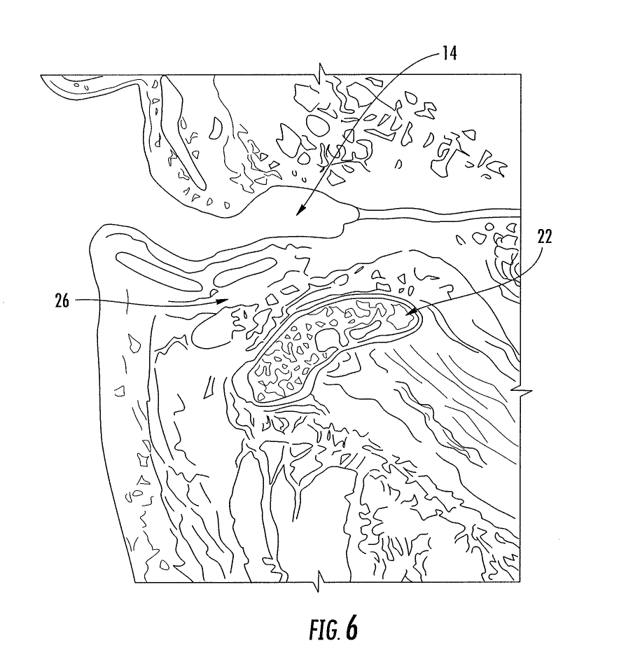

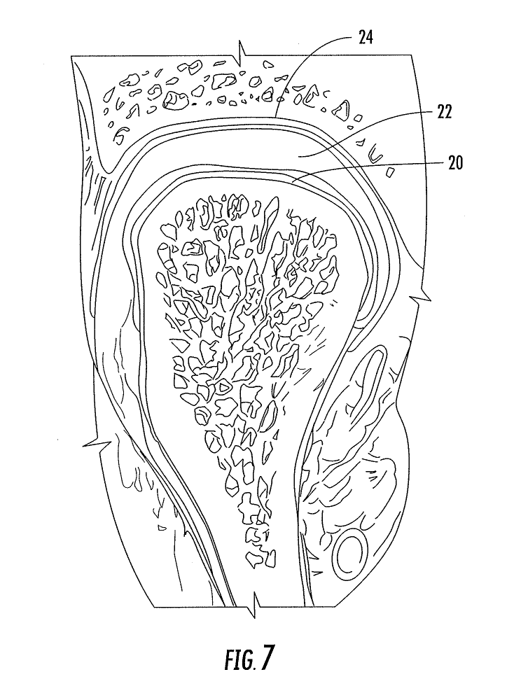

The TMJ is positioned in front of the ear canal, as illustrated in FIGS. 2 and 6. Disclosed herein are various applications that capitalize on the TMJ's proximity to the ear canal to influence the operation of the TMJ and to help alleviate or prevent symptoms and discomfort associated with TMD.

In-Ear Proprioceptive

Disclosed is an in-ear device (a proprioceptive) having one or more proprioceptive features for alleviating or reducing one or more TMJ-related symptoms in a subject. As used herein, proprioception refers to a conscious or subconscious indication to a subject that influences the subject's perception and, in some cases, the subject's behavior. In some instances, proprioception influences a subject's behavior even if the subject is not consciously aware of it. In particular, in some embodiments, the in-ear device includes one or more features that influence the subject's perception. In some embodiments, the one or more features provide one or more proprioceptive cues or indicators to the subject informing the subject to alter his movements to avoid or reduce pain associated with the TMJ and/or to avoid or reduce deterioration of the TMJ. As described in more detail below, these indicators can be passive or active or any suitable combination of both.

In-Ear Proprioceptive with Active Indicators

In some embodiments, the one or more proprioceptive features are mechanical and/or electrical sensors. These sensors may be referred to as active indicators. One non-limiting embodiment of an in-ear device 90 with at least one sensor component 92 is shown in FIG. 12. As shown, the sensor component 92 may include a proprioceptive feedback component 88, a processor 90, one or more sensors 92, random access memory (RAM) 94, and a threshold module 96. Sensor 92 may include any number of sensors and may be any suitable sensor, such as a force sensor, an accelerometer, a voltage sensor, and/or any other suitable sensor. In one non-limiting embodiment, the one or more sensors 92 measures a physical quantity and sends information associated with the physical quantity to processor 90. The processor 90 uses information stored in the threshold module 96 to determine if the physical quantity exceeds a predetermined threshold stored in memory. If the physical quantity exceeds the predetermined threshold, the processor 90 instructs the proprioceptive feedback component 88 to generate a suitable signal. In this embodiment, the actor in this active configuration is a software module that compares the value received from the sensor to the threshold held in memory and determines whether to send a proprioceptive signal.

In some embodiments, proprioceptive feedback component 88 is a vibration motor or speaker or any other component capable of generating a suitable signal or earcon to the subject as discussed below. In some embodiments, the sensor component 92 is an analog system that does not require a processor or memory.

In one embodiment, the sensor 92 may be a force sensor that is configured to measure the force exerted by the jaw to determine when the jaw is being clenched and/or the teeth are grinding or the jaw has otherwise moved too far and the subject is approaching the point of TMJ-related symptoms (e.g., pain or discomfort). Specifically, when the jaw is clenched and/or the teeth are grinding, the shape and/or position of the subject's ear canal changes, inherently exerting a force on the in-ear device. The force sensor can be used to measure the force exerted on the in-ear device when the jaw is clenched and/or the teeth are being ground. The in-ear device then can be programmed so that, when the force sensor detects force approaching this predetermined measurement in use, a transducer transmits an appropriate signal to the subject.

The signal generated by the proprioceptive feedback component 88 may be a vibration, an audio signal, or any other suitable signal that indicates to a subject that he is clenching/grinding his teeth and that he is approaching the point of invoking TMJ-induced symptoms and/or deterioration. In some embodiments, the signal is generated when the subject's jaw is clenched or he is grinding his teeth, or when he closes his jaw past a predetermined threshold/limit of movement. In some embodiments, the predetermined limit of movement corresponds to the subject's jaw position associated with one or more symptoms of TMD.

In some cases, the force sensor alone may be incapable of detecting movement of the jaw past the predetermined threshold and therefore may be insufficient to provide the desired feedback to the subject. In these situations, the sensor 92 may include an accelerometer (instead of or in addition to) the force sensor that monitors the rate of motion of the jaw. When the acceleration of the jaw exceeds a certain threshold (such as when the jaw is clenched and/or opened too wide or otherwise moved to an extreme point with sufficient acceleration), the accelerometer can send a signal to the subject indicating that the rate of change in the jaw position needs to be changed to avoid or reduce one or more TMJ-related symptoms. The accelerometer also may be configured to detect joint sounds and provide feedback based on the detected joint sounds.

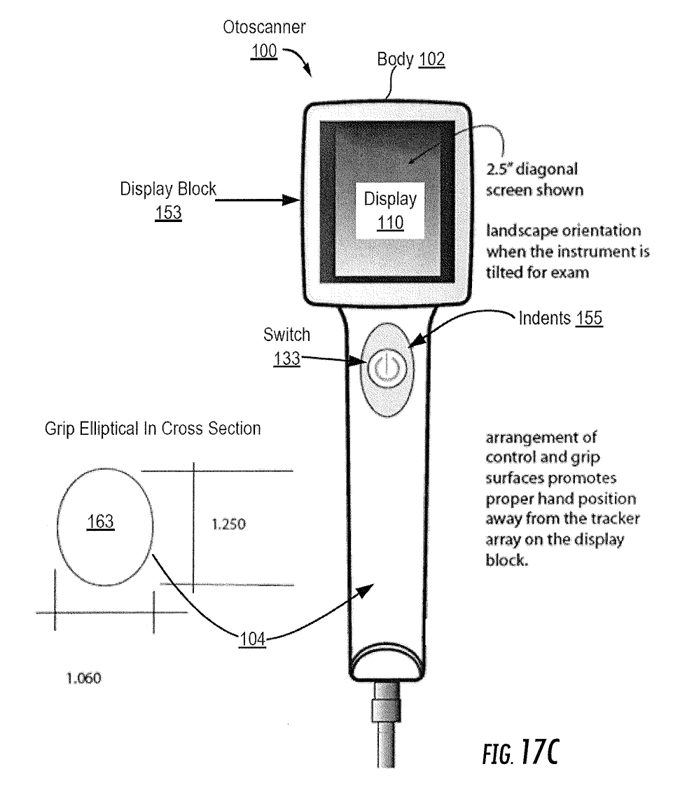

As shown in FIG. 16, the mandibular branch (V3 branch) 34 of the trigeminal nerve runs near the TMJ. The mandibular innervates the muscles involved in mastication (chewing). During clenching and grinding, which as described above are factors that cause TMJ dysfunction, these muscles are activated through efferent electrical signals through the mandibular nerve. In one embodiment, the sensor 92 may be a voltage detector that measures the voltage across the mandibular nerve from inside the ear canal. When the voltage reaches a predetermined threshold, the device transmits a signal (such as an auditory signal or a vibration or other suitable signal) to the subject. The voltage detector may also be used to measure the voltage across a muscle (such as the masseter, temporalis, or pyerygoid muscles) to determine whether the subject is clenching or grinding.

In some embodiments, the in-ear device does not include an input signal, but is configured to emit a signal that is time dependent. For example, the in-ear device can be configured to send a signal to the subject at predetermined intervals. For example, a vibration, audio, or other suitable signal emitted at predetermined temporal intervals may provide a subject with feedback to consciously assess and correct the positioning of his jaw to relieve stress on the TMJ and reduce inflammation and deterioration.

In some embodiments, these active proprioceptive mechanical and/or electronic indicators replace one or more passive proprioceptive features described below. In other embodiments, these mechanical and/or electronic signals are used in addition to the one or more passive proprioceptive indicators described below. In each case, the features are selected to meet the particular needs of the subject.

In-Ear Proprioceptive with One or More Passive Indicators

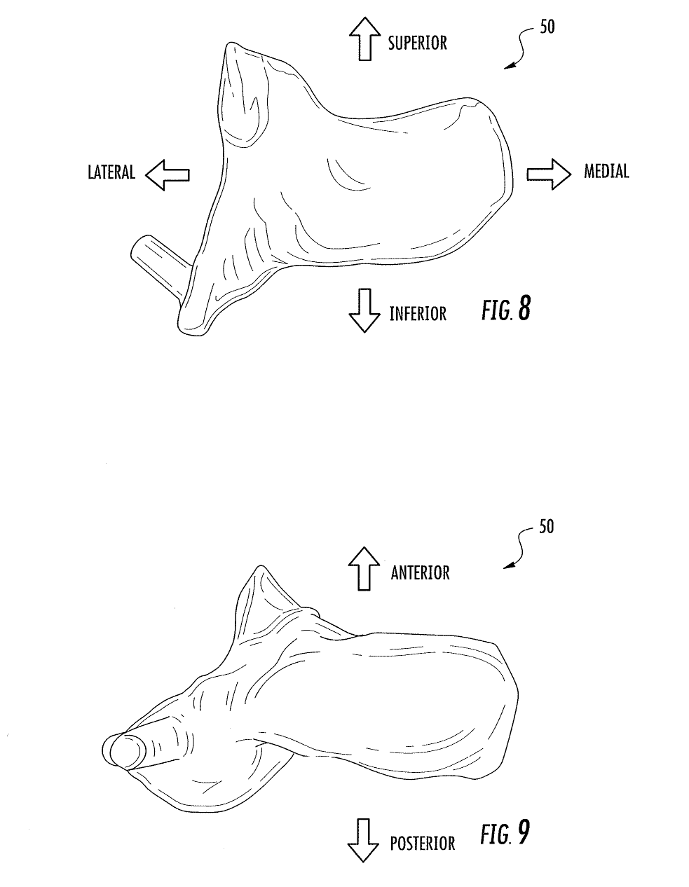

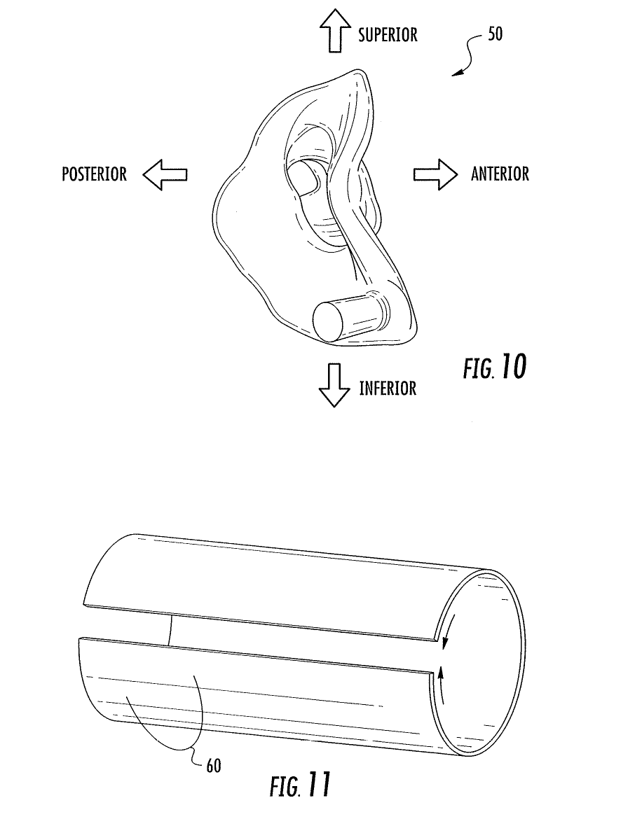

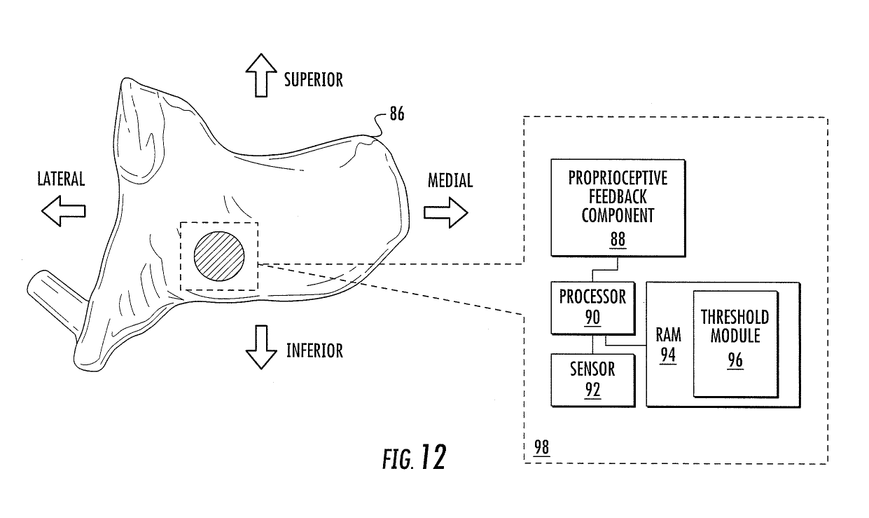

In some embodiments, the in-ear device is custom-designed so that it substantially conforms to a particular subject's ear canal when the jaw is in a particular location and/or so that it deforms the subject's ear canal when the jaw moves in a predetermined way. A non-limiting example of a custom-designed in-ear device is shown in FIGS. 8-10 as in-ear device 50.

Generally, the cross-sectional area and configuration of the ear canal changes as a subject opens and closes or otherwise moves his jaw. In addition, the ear canal may translate in any direction as the subject moves his jaw. With some people, the cross-sectional area of the ear canal decreases as the jaw moves from its therapeutic or optimal position to the closed position and/or as the jaw moves from its therapeutic position or optimal to the open position. Moreover, with some subjects, the subject's jaw moves in an anterior-posterior and/or superior/inferior direction as the subject's jaw moves from its therapeutic or optimal position.

The therapeutic or optimal position of the jaw is one that changes a subject's symptomatic and/or dysfunctional maxillomandibular relationship to one that is more normal, less symptomatic and/or more fully functional, and in some cases involves repositioning the mandible vertically, anteroposteriorly and/or transversely to the extent necessary. The therapeutic or optimal position of the jaw varies from subject to subject, but can be determined using any suitable, conventional method, some examples of which are given below. In some cases, the therapeutic or optimal position is a neutral, more asymptomatic position of the jaw that helps relieve stress on the TMJ disc and surrounding tissues. In some cases, the therapeutic position is between an extreme closed position and an extreme open position of the jaw and is a position that reduces one or more symptoms of the temporomandibular joint disorder. It is within the skill of one of skill in the art to select the therapeutic or optimal jaw position for any given subject.

For some subjects, an in-ear device situated within the ear canal will mechanically exert forces on the ear canal when the cross-sectional area of the ear canal decreases and/or when the ear canal translates, providing proprioceptive cues. When the cross-sectional area of the ear canal decreases beyond a predetermined value, the forces exerted on the ear canal as the in-ear device deforms the ear canal may be sufficient to provide an indication (such as a sensation of discomfort or fullness in the ear canal) to the subject that he has closed (or opened) his mouth or otherwise moved his jaw to the selected TMJ threshold, and that he should stop movement to avoid or reduce one or more TMJ-related symptoms and/or inflicting further damage on the TMJ.

In some embodiments, the in-ear device is configured and/or dimensioned so that the forces exerted on the ear canal are sufficient to provide the subject with the sensory indication when the subject begins clenching/grinding his teeth and/or when he closes his jaw beyond a predetermined threshold. In this way, the device itself is configured to have a proprioceptive feature that functions to provide mechanical resistance and alert a subject to alter the movement of his jaw to prevent or reduce TMJ-related symptoms and/or deterioration. This proprioceptive feature is sometimes referred to as a passive indicator.

In some cases, continuous pressure or regular proprioception causes the subject's muscles to relax (either through proprioception or through pressure caused by deformation of the ear canal). Moreover, in some cases, deforming the subject's ear canal or otherwise using an in-ear device to exert pressure on the ear canal may help relieve pain associated with TMD. According to a theory known as the Gate Theory, activating diameter nerve fibers by grabbing, holding, applying pressure to, and/or rubbing a painful site can inhibit (suppress) pain sensation at the spinal cord level from that segment of the body. As such, the in-ear devices described herein can be used to apply pressure in a way that reduces pain or other symptoms associated with TMD.

The in-ear device may be used in one or both ears depending on the needs of the subject. In some embodiments, the in-ear device is customized to conform to a particular subject's unique ear canal, as discussed below.

The in-ear device may be formed of any suitable material, such as, but not limited to, polymers such as polypropylene (PP), polyethylene (PE), polytetrafluoroethylene (PTFE), acrylic, acrylonitrile budadiene styrene (ABS), polyether ether ketone (PEEK), silicone, thermoplastic elastomers such as polyurethane, or any other suitable material. In some cases, the material is selected so the in-ear device is capable of being compressed for insertion into the ear and so that the in-ear device expands to its original state after a predetermined period of time. In some embodiments, the in-ear device is formed of a heat-dependent shape memory polymer or alloy. One non-limiting example is a nickel titanium alloy (nitinol).

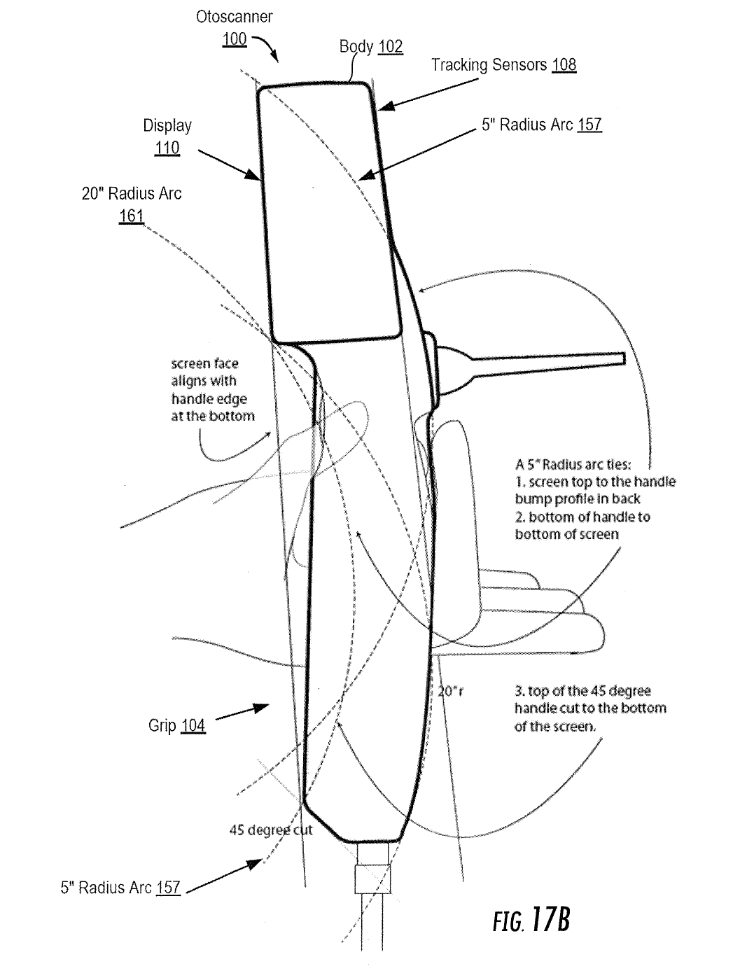

As mentioned, the in-ear device may be formed of any suitable material, including, for example, a combination of rigid and soft materials, as shown in FIG. 15. The in-ear device may have any suitable durometer, for example, a durometer between approximately 20 A-80 A. The durometer of the device can be customized based on the particular hardness and elasticity of the subject's ear canal. In some embodiments, subsurface imaging or any other suitable technique may be used to determine the hardness and elasticity of the subject's ear canal. In the non-limiting embodiment illustrated in FIG. 15, the in-ear device is formed of a combination of rigid and soft materials. For example, the inner material 82 may be a rigid or semi-rigid material (e.g., but not limited to, a material having a durometer of approximately 60 A-80 D) that provides support to the in-ear device, while the outer material 84 may be a relatively soft and flexible material (e.g., but not limited to, a material having a durometer of approximately 10 OO-40 A) that is relatively comfortable when in contact with the subject's ear canal. In some embodiments, the in-ear device has a hollow center 80, as shown in FIG. 15.

The combination of materials may also be selected so that the in-ear device selectively expands. In particular, the materials may be selected so that the device only expands in portions that correspond to areas of the ear canal where deformation is desired (i.e., where it is desired that the forces supplying the sensory indication be supplied). The rigidity of the material can also be selected to limit TMJ motion, as an increase in rigidity limits more motion than a less rigid or relatively soft material.

In some embodiments, as shown in FIG. 11, the in-ear device may be generally C-shaped or have a generally C-shaped internal cavity or sound channel. A generally C-shaped device as shown in FIG. 11 may facilitate compression of the in-ear device before insertion and therefore facilitate the insertion of the device into the ear canal. The split C-shaped nature of the device illustrated in FIG. 11 may also be configured to help direct the forces associated with the sensory indication to the subject. In some cases, the split nature of the device provides a spring-like effect that helps orient the device properly within the ear canal. The generally C-shaped device is optionally customized to conform to the subject's ear canal, thus providing a device that is customized, but is easily insertable. As one non-limiting example, the flexural modulus of the generally C-shaped device may be selected to vary how much force the device applies to the ear canal.

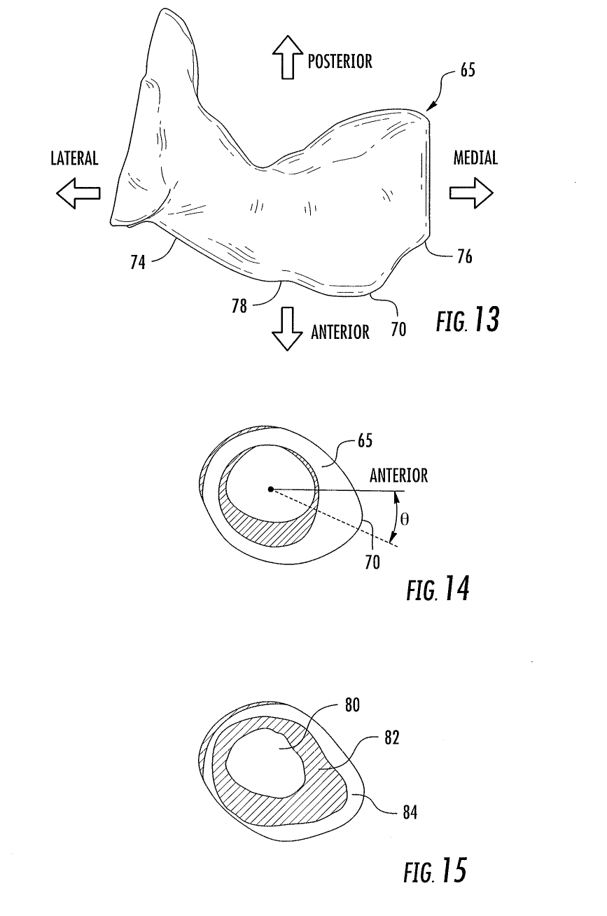

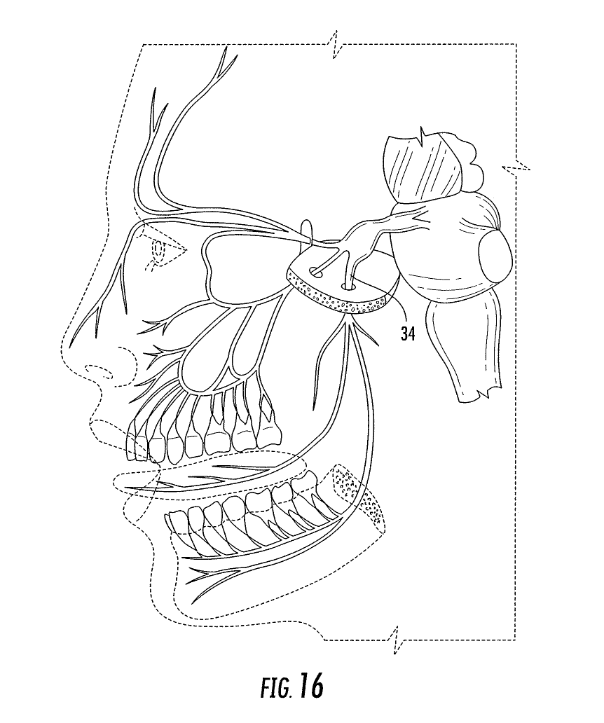

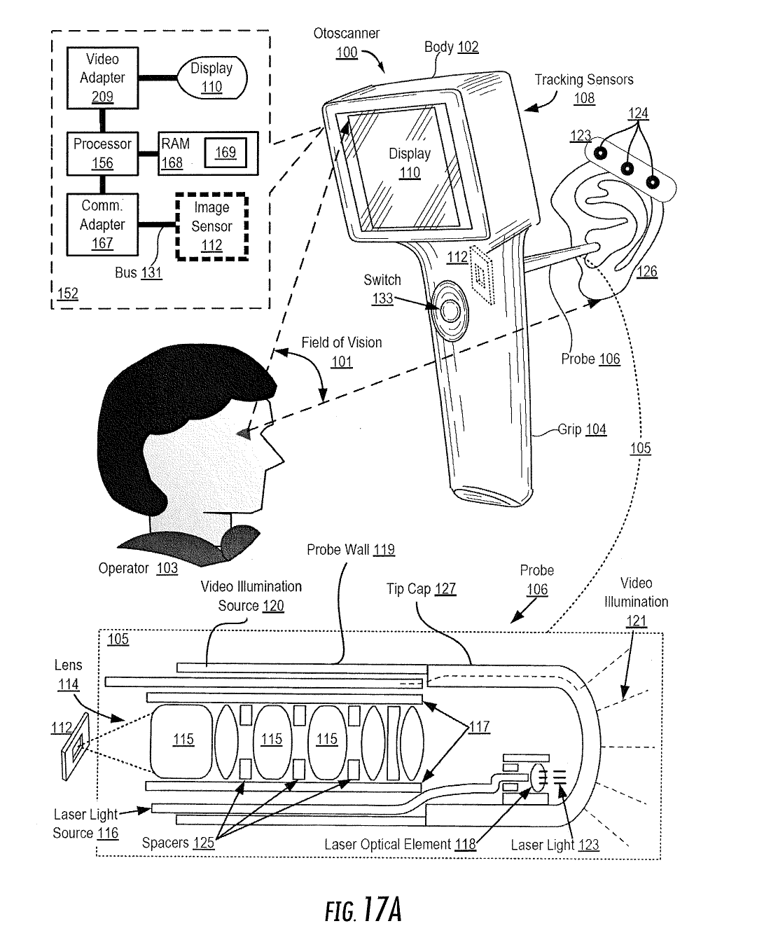

In some embodiments, the in-ear device includes a protrusion 60 that protrudes from the device, an embodiment of which is shown in FIG. 11, or a protrusion 70 as shown in FIG. 13. Generally, the protrusion may be positioned along the in-ear device at a customizable relative distance. For example, the protrusion may be positioned along the in-ear device such that it is situated within either the first bend 16 or the second bend 18 of the ear canal 12 when the device is inserted in the ear canal 14. As shown in FIG. 13, protrusion 70 may be positioned along in-ear device 65 a predetermined distance from any suitable landmark such as the first bend 78, the second bend 76, or the aperture 74.

In some cases, the protrusion 60 is configured to project from the in-ear device at a predetermined angle that corresponds to the configuration of the particular subject's ear canal. In this way, along with the location of the protrusion along the in-ear device, the angle θ (see FIG. 14) from which the protrusion projects from the in-ear device may be customized based on the particular subject's ear canal.

In some embodiments, more than one protrusion is included. In some cases, the first protrusion is positioned along the device such that it is situated within the first bend of the ear canal when the in-ear device is inserted in the ear canal and the second protrusion is positioned along the device such that it is situated within the second bend of the ear canal when the device is inserted in the ear canal.

Alternatively, the one or more protrusions may be positioned at any other suitable location along the in-ear device depending on the configuration of the particular subject's ear canal. For example, the protrusion 60 may be positioned along the in-ear device so that it is situated within the portion of the particular subject's ear canal that expands/contracts the most throughout the jaw movement (i.e., the segment of the canal with the most mobility). Because in these embodiments the protrusion 60 is situated within the portion of the ear canal with the most expansion/mobility, a sensory indication is provided to the subject based on the forces exerted by the protrusion 60 when the subject begins to clench/grind his teeth or has otherwise reached his jaw's threshold for opening and/or closing or other movement. In some cases, the protrusion is also referred to as a passive indicator, as it is the interaction of the in-ear device itself with the ear canal that provides the sensory indication.

Optionally, the protrusion includes a durometer, which may be selected so that it has a rigidity sufficient to exert force on the ear canal when the subject is grinding/clenching his teeth and/or his jaw is opened too wide or otherwise moved too far and so as to provide a sensory indication to the subject to alter the movement of his jaw to avoid or reduce one or more TMJ-related symptoms. The durometer of the protrusion may be customized based on the configuration of the particular subject's ear canal and the sensitivity of his sensory receptors. In some non-limiting embodiments, the durometer of the protrusion is between approximately 60 A-80 D.

In some cases, the protrusion is added if the forces exerted by the in-ear device are insufficient to provide the particular subject with a sensory indication that he should limit his jaw's movement or if more precise control is needed or desired. Depending on the needs of the subject, the in-ear device can include any suitable number and type of passive and/or active indicators. In some embodiments, the in-ear device does not include any passive or active indicators, but is customized based on the particular subject's ear canal to deform the subject's ear canal in a way that alleviates one or more symptoms of TMD.

Method of Designing a Custom in-Ear Orthotic

As shown in FIGS. 8-10, the in-ear orthotic may be one that is customized based on the particular subject's ear canal. In this way, the in-ear orthotic conforms to at least a portion of the particular subject's ear canal. The customized orthotic can be designed using any suitable method, such as, but not limited to, scanning the ear canal to create a 3D replication of the ear canal. In some cases, the in-ear orthotic may also be customized based on scans of the outside of the jaw. For example, U.S. Ser. No. 13/417,767, filed Mar. 12, 2012 and titled “Optical Scanning Device”; Ser. No. 13/417,649, filed Mar. 12, 2012 and titled “Otoscanning with 3D Modeling”; Ser. No. 13/586,471, filed Aug. 15, 2012 and titled “Video Otoscanner with Line-of-Sight of Probe and Screen”; Ser. No. 13/586,411, filed Aug. 15, 2012 and titled “Otoscanner with Fan and Ring Laser”; Ser. No. 13/586,459, filed Aug. 15, 2012 and titled “Otoscanner with Camera for Video and Scanning”; Ser. No. 13/586,448, filed Aug. 15, 2012 and titled “Otoscanner with Pressure Sensor with Compliance Measurement”; and Ser. No. 13/586,474, filed Aug. 15, 2012 and titled “Otoscanner with Safety Warning System,” the contents of all of which are incorporated herein by reference in their entireties, disclose suitable methods of scanning the ear canal, building a three-dimensional shape, and designing a customized in-ear orthotic based on the generated three-dimensional shape.

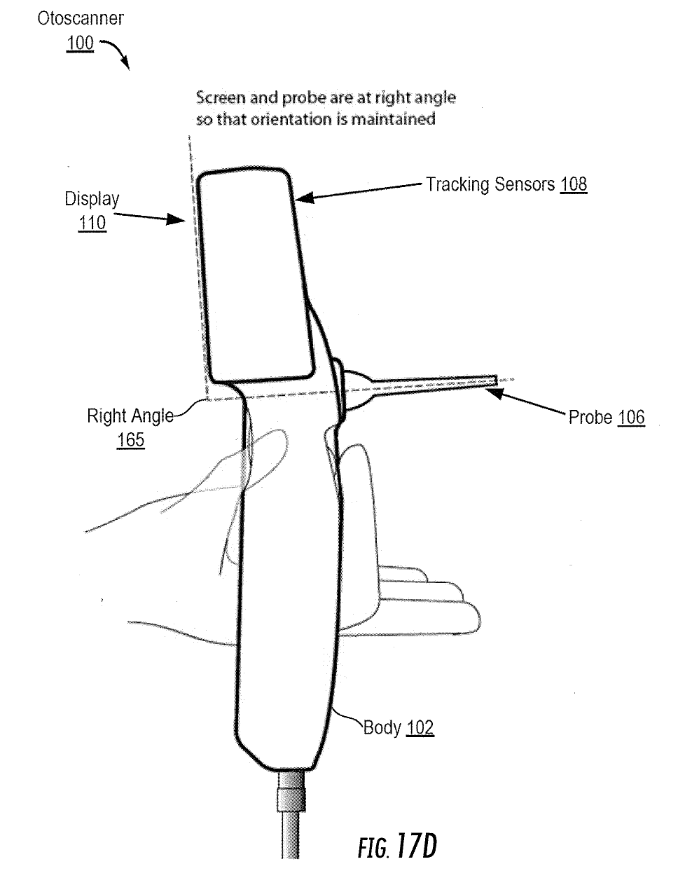

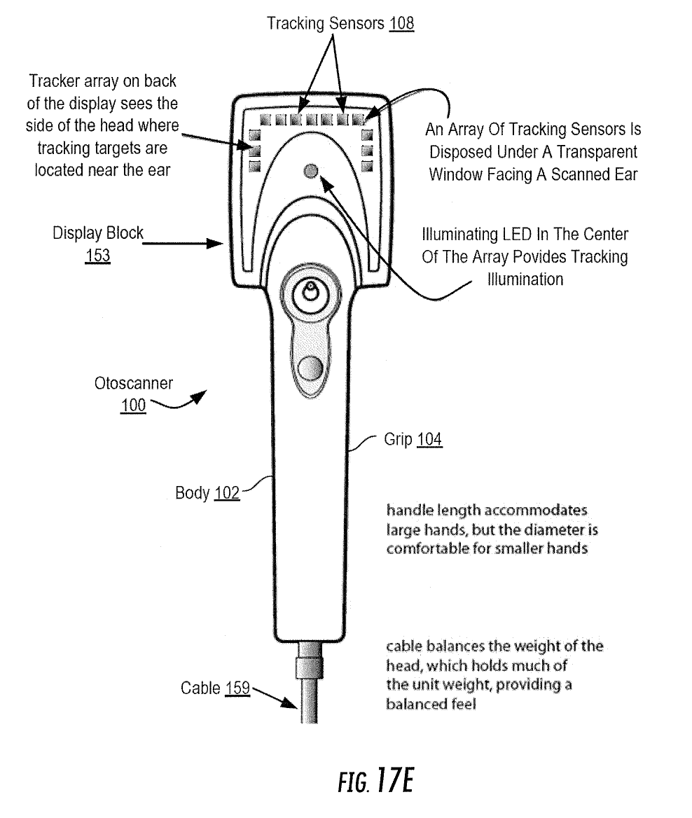

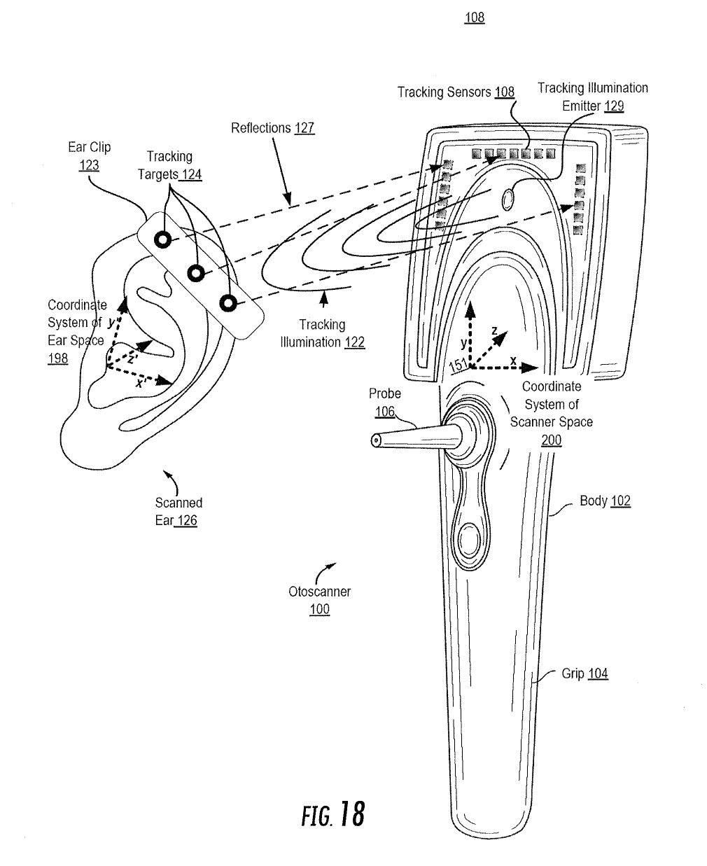

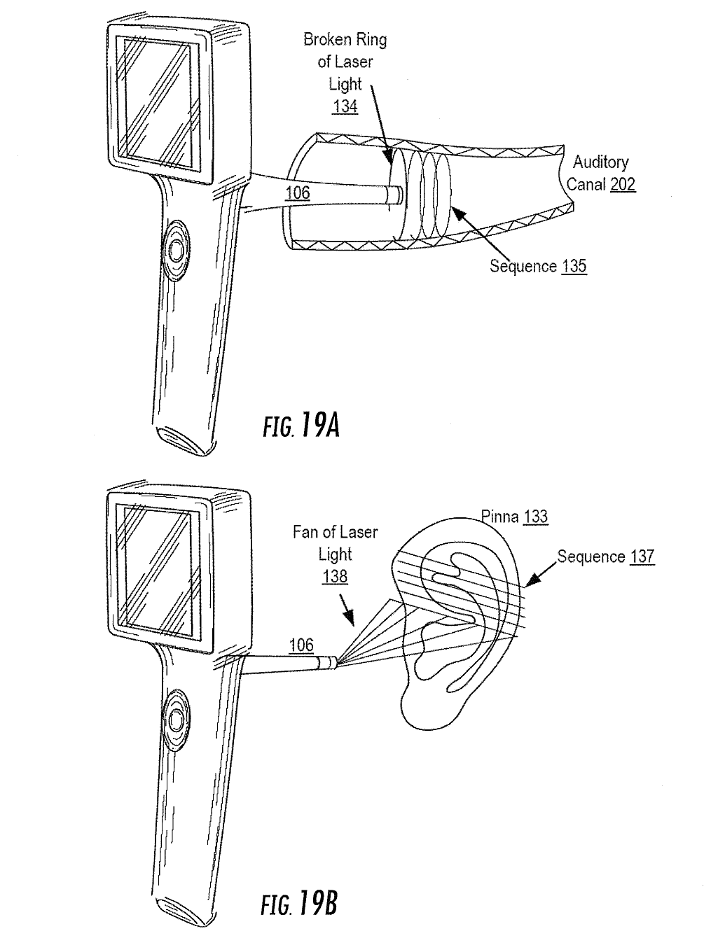

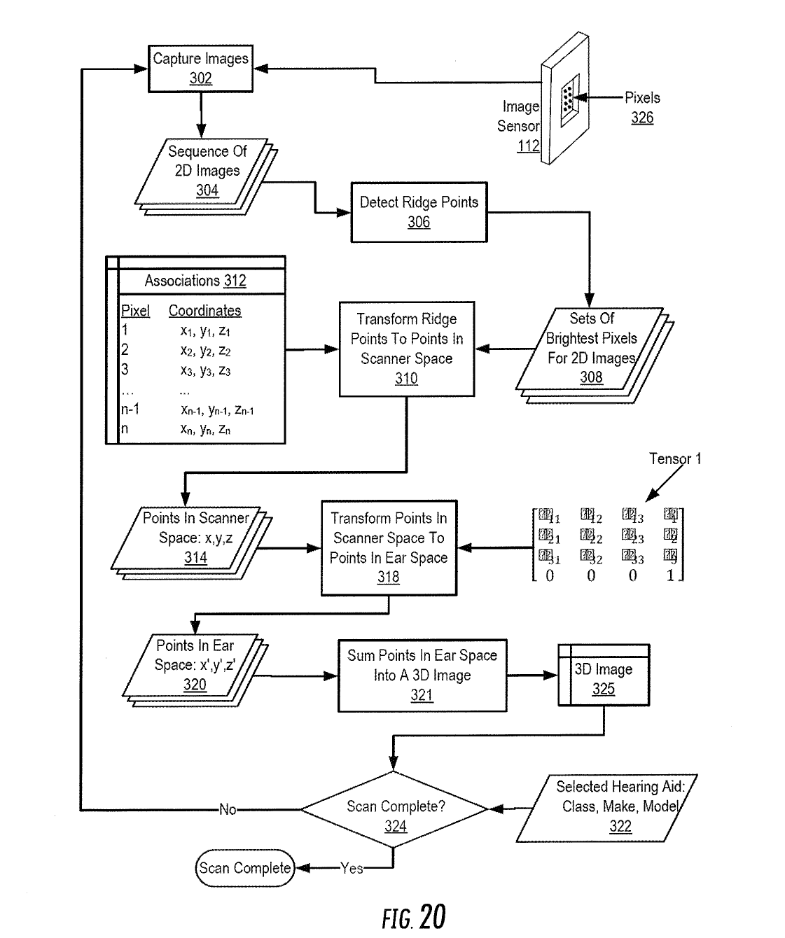

Example scanning apparatus and methods according to some embodiments are described with reference to the accompanying drawings, beginning with FIG. 17. FIG. 17A sets forth a line drawing of an example scanner 100 having a scanner body 102. The scanner body 102 includes a hand grip 104. The scanner body 102 has mounted upon it an ear probe 106, a tracking illumination emitter 129 (FIG. 18), a plurality of tracking illumination sensors 108 (not visible on FIG. 17A but visible on FIG. 18), and a display screen 110. The scanner body has mounted within it an image sensor 112.

The display screen 110 is coupled for data communications to the image sensor 112, and the display screen 110 displays images of the scanned ear 126. FIG. 17A includes a callout 152 that schematically illustrates an example of the display screen 110 coupled for data communications to the image sensor 112 through a data communications bus 131, a communications adapter 167, a data processor 156, and a video adapter 209. The displayed images can include video images of the ear captured by the image sensor 112 as the probe is moved within a scanned ear 126. The displayed images can include real-time constructions of 3D images of the scanned ear, such as the one illustrated on FIG. 29. The displayed images can also include snapshot images of portions of the scanned ear.

In the example of FIG. 17A, the display screen 110 is positioned on the scanner body 102 in relation to the ear probe 106 so that when the ear probe 106 is positioned for scanning, both the display screen 110 and the ear probe 106 are visible to any operator 103 of the scanner 100. In one embodiment, the scanner 100 is implemented with the ear probe 106 mounted on the scanner body 102 between the hand grip 104 and the display screen 110 mounted on the opposite side of the scanner body 102 from the ear probe 106 and distally from the hand grip 104. In this way, when an operator takes the grip in the operator's hand and positions the probe to scan an ear, both the probe and the display are easily visible at all times to the operator.

In some embodiments, the display screen 110 is not positioned on the scanner body 102 in any particular relation to the ear probe 106. That is, in some such embodiments, during scanning the ear probe is not visible to the operator or the display screen is not visible to the operator. The ear probe may therefore be located anywhere on the scanner body with respect to the display screen if both are integrated into the scanner. And furthermore, in some embodiments, the scanner may not even have an integrated display screen.

FIG. 17A includes a callout 105 that illustrates the ear probe 106 in more detail. The ear probe 106 includes a wide-angle lens 114 that is optically coupled to the image sensor 112, with the lens and the sensor oriented so as to capture images of surfaces illuminated by light from laser and non-laser light sources in the probe. In the example scanner probe 106 of FIG. 17A, the wide angle lens 114 has a sufficient depth of field so that the entire portion of the surface of an ear 126 illuminated by laser light is in focus at the image sensor 112. An image of a portion of the scanned ear is said to be in focus if light from object points on the surface of the ear is converged as much as reasonably possible at the image sensor 112, and out of focus if light is not well converged. The term “wide angle lens” as used herein refers to any lens configured for a relatively wide field of view that will work in tortuous openings such as an auditory canal. For example, for an auditory canal, a 63 degree angle results in a lens-focal surface offset about equal to the maximum diameter of the auditory canal that can be scanned with a centered ear probe. The focal surface of a 60 degree lens (a fairly standard sized wide angle lens) is equal to the diameter, resulting in a forward focal surface of about 6 mm, which typically is short enough to survive the second bend in an auditory canal which is at about a 6 mm diameter. For scanning auditory canals, therefore, wide angle lenses typically are 60 degrees or greater. Other functional increments include 90 degrees with its 2:1 ratio allowing a forward focal surface distance of about 3 mm, allowing an ear probe to be fairly short. Lenses that are greater than 90 degrees are possible as are lenses that include complex optical elements with sideways only views and no forward field of view. According to some embodiments, laser light is emitted from the scanner probe in the form of a ring or in the form of a fan, and the wide angle lens provides the same sufficient depth of field to portions of a scanned ear as illuminated by all such forms of laser.

The wide angle lens 114 can view relatively proximate lateral portions of a surface with high precision due to overlap of its focal surface with a pattern of projected laser light. The term “focal surface” refers to a thickness within a range of focus of the wide angle lens that is capable of achieving a certain base line resolution, such as being able to discern a 50 micrometer feature or smaller. In an embodiment, for example, lateral positioning of a pattern of projected laser light within the focal surface can allow one pixel to be equivalent to about 50 micrometers. Such a focal surface itself would have a bell curve distribution of resolution that would allow variations in overlap or thickness of the focal surface and the width of the lateral portion of reflected laser light which, as described in more detail below, has its own curved distribution across its thickness.

Wide angle lenses 114 in embodiments typically have a reasonably low distortion threshold to meet resolution goals. Most wide angle lenses can be as high as −80 percent or −60 percent distortion that would need to be compensated by improved accuracy in other areas such as placement of the focal surface and lateral portion of projected patterns of laser light. There is therefore no set threshold although collectively the various components are preferably tuned to allow a 50 micrometer or better resolution for lateral distances from the optical axis of the wide angle lens. A distortion of −40 percent or better provides a workable field of view for scanning auditory canals.

The ear probe 106 includes a laser light source 116, a laser optical element 118, and a source of non-laser video illumination 120. The laser light source 116 delivers laser light 123 that illuminates surfaces of a scanned ear 126 with laser light, and the video illumination source delivers video illumination that illuminates surfaces of a scanned ear with non-laser light 121. In the example of FIG. 17A, the laser light source 116 in the ear probe is implemented as an optical fiber 130 that conducts laser light to the ear probe 106 from a laser outside the probe 106. In fact, in the example of FIG. 17A, both sources of illumination 116, 120 are implemented with optical fiber that conduct illumination from, for example, sources mounted elsewhere in the scanner body, a white light-emitting-diode (‘LED’) for the non-laser video illumination 121 and a laser diode or the like for the laser light 123. For further explanation, an alternative structure for the laser light source is illustrated in FIG. 22, where the laser light source is implemented as an actual laser 158, such as, for example, an on-chip laser diode, mounted directly on mounting structures disposed in the probe itself. In the example of FIG. 22, a laser power source 160, electrical wiring, replaces the optical fiber 116 (FIG. 17A) in the overall structure of the probe, connecting a power supply outside the probe to the laser 158. In the examples both of FIG. 17A and FIG. 22, the laser light 123 is collimated by a laser optical element 118, and the non-laser video illumination 121 is diffused by a transparent top cap 127 mounted on the tip of the probe. Laser illumination from the laser light source 116 can be on continuously with the LED pulsed or both the laser and the LED can be pulsed, for example.

The scanner 100 in the example of FIG. 17A provides a mode switch 133 for manual mode switching between laser-only mode, in which a laser-illuminated scan of an ear is performed without video, and a video-only mode in which non-laser light is used to illuminate a scanned ear and normal video of the ear is provided on the display screen 110. The laser light may be too bright to leave on while capturing video images, however, so with manual switching, only one mode may be employed at a time. In some embodiments of the kind of scanner illustrated for example in FIG. 17A, therefore, the image sensor is configured so as to capture images at a video frame rate that is twice a standard video frame rate. The frame rate is the frequency at which an imaging sensor produces unique consecutive images called frames. Frame rate is typically expressed in frames per second. Examples of standard video frame rates include 25 frames per second as used in the Phase Alternating Line or ‘PAL’ video standard and 30 frames per second as used in the National Television System Committee or ‘NTSC’ video standard. At twice a standard frame rate, video and laser-illuminated images can be captured on alternate frames while leaving the frame rate for each set to a standard video rate. In such embodiments, the non-laser video illumination 120, 12) is left on at all times, but the laser light source 116 is strobed during capture by the image sensor of alternate video frames. Video frames are captured by the image sensor 112 when only the non-laser video illumination illuminates the scanned ear, that is, on the alternate frames when the laser light source 116 is strobed off. Then laser-illuminated images for constructing 3D images are captured by the image sensor 112 only when strobed laser light illuminates the scanned ear, that is, during the alternate frames when the laser light source 116 is strobed on, overwhelming the always-on non-laser video illumination.

For further explanation, FIGS. 17B-17E set forth line drawings of further example scanners, illustrating additional details of example embodiments. In the example of FIG. 17B, a scanner 100 includes a body 102, display 110, tracking sensors 108, and grip 104, all implemented in a fashion similar to that of the scanner describes and illustrated above with reference to FIG. 17A. The example of FIG. 17B includes 5-inch radius arcs 157 that defines and connect the screen top to a grip bump profile on the back of the scanner body, the bottom of the grip to the bottom of the display screen, and the top of a 45-degree cut at the bottom of the grip to the bottom of the display screen. In addition, the example of FIG. 17B includes a 20-inch radius arc 161 that defines the overall curvature of the grip 104.

In the example of FIG. 17C, a scanner 100 includes a body 102, display 110, tracking sensors 108, and grip 104, all implemented in a fashion similar to that of the scanner described and illustrated above with reference to FIG. 17A. The example of FIG. 17C includes a description of the grip 104 as elliptical in cross section, conforming to an ellipse 163 in this example with a major axis 1.25 inches in length and a minor axis of 1.06 inches. The example of FIG. 17C also includes a display screen 2.5 to 3.5 inches, for example, in diagonal measure and capable of displaying high-definition video. The display screen 110 is also configured with the capability of displaying images in portrait orientation until the scanner body is oriented for scanning an ear, at which time the display can change to a landscape orientation. Indents 155 are provided around control switches 133 both on front and back of the grip 104 that guide operator fingers to the control switches with no need for an operator takes eyes off the display screen or the probe to look for the switches.

In the example of FIG. 17D, a scanner 100 includes a body 102, display 110, tracking sensors 108, and grip 104, all implemented in a fashion similar to that of the scanner described and illustrated above with reference to FIG. 17A. The example of FIG. 17D includes an illustration of the display screen 110 oriented at a right angle 165 to a central axis of the ear probe 106 so as to maintain the overall orientation of the display as it will be viewed by an operator.

In the example of FIG. 17E, a scanner 100 includes a body 102, tracking sensors 108, and grip 104, all implemented in a fashion similar to that of the scanner described and illustrated above with reference to FIG. 17A. The example of FIG. 17E includes an illustration of the orientation of an array of tracking sensors 108 on the back of the display, that is, on the opposite side of the scanner body from the display screen, oriented so that the tracking sensor can sense reflections of tracking illumination from tracking targets fixed in position with respect to a scanned ear. The tracking sensor are disposed behind a window that is transparent to the tracking illumination, although it may render the tracking sensors themselves invisible in normal light, that is, not visible to a person. The example of FIG. 17E also includes a grip 104 whose length accommodates large hands, although the diameter of the grip is still comfortable for smaller hands. The example of FIG. 17E also includes a cable 159 that connects electronic components in the scanner body 102 to components outside the body. The cable 159 balances the weight of the display block, which holds much of the weight of the scanner body. The use of the cable 159 as shown in FIG. 17E provides to an operator an overall balanced feel of the scanner body.

Referring again to FIG. 17A, the image sensor 112 is also coupled for data communications to a data processor 128, and the data processor 128 is configured so that it functions by constructing, in dependence upon a sequence of images captured when the scanned ear is illuminated by laser light and tracked positions of the ear probe inferred from reflections of tracking illumination sensed by the tracking illumination sensors, a 3D image of the interior of the scanned ear, such as, for example the image illustrated in FIG. 29. For further explanation, FIG. 18 sets forth a line drawing of an example scanner with a number of tracking illumination sensors 108 disposed upon the scanner body 102 so as to sense reflections 127 of tracking illumination 122 emitted from the tracking illumination emitter 129 and reflected from tracking targets 124 installed at positions that are fixed relative to the scanned ear 126. The tracking illumination sensors 127 are photocells or the like disposed upon or within the opposite side of the display block from the display and organized so as to distinguish angles and brightness of tracking illumination reflected from tracking targets. In the example of FIG. 18, the tracking targets 124 are implemented as retroreflectors, and the tracking illumination 122 is provided from a tracking illumination source or emitter 129, such as an LED or the like, mounted on the scanner body 102. In at least some embodiments, the tracking illumination 122 is infrared.

In the example of FIG. 18, the tracking sensors 108 are mounted directly on or within the scanner 100. In other embodiments, the tracking sensors are mounted elsewhere, in other locations fixed within scanner space, not on or within the scanner itself. In such embodiments, a stand alone or separate tracking system can be used. Such embodiments can include one or many tracking sensors, one or many light sources. Some embodiments exclude tracking entirely, instead relying of the stability of an object to be scanned. To the extent that such an object is an ear, then the person to whom the ear belongs must sit very still during the scan. Other embodiments use a tripod for mounting the tracking systems of tracking illumination sensors.

The data processor 128 is configured so that it constructs a 3D image of the interior of the scanned ear can be implemented, for example, by a construction module 169 of computer program instructions installed in random access memory (‘RAM’) 168 operatively coupled to the processor through a data communications bus. The computer program instructions, when executed by the processor, cause the processor to function so as to construct 3D images based on tracking information for the scope body or probe and corresponding images captured by the image sensor when a surface of a scanned ear is illuminated with laser light.

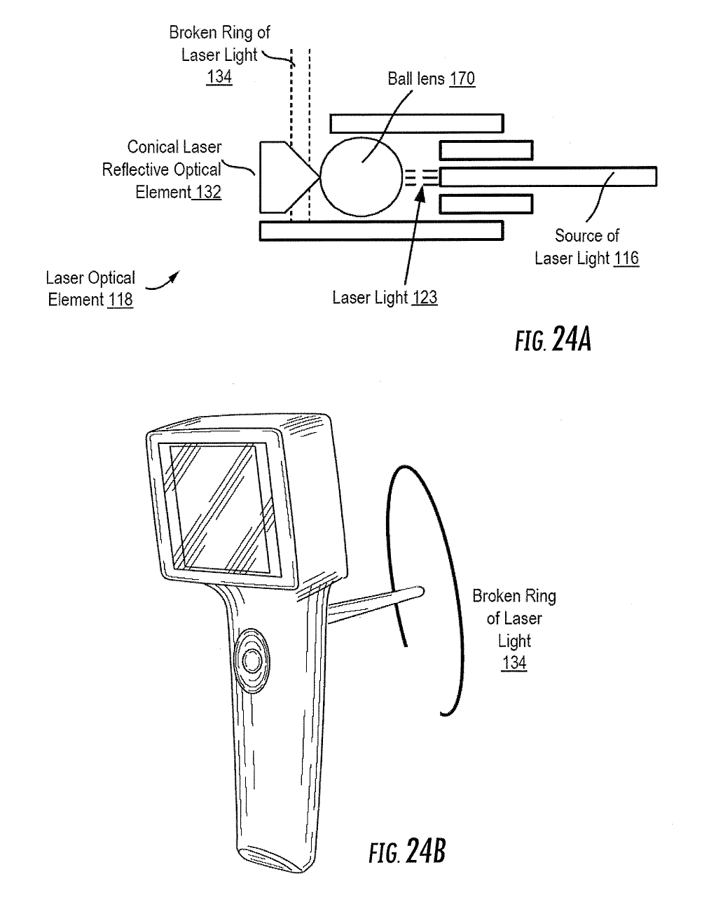

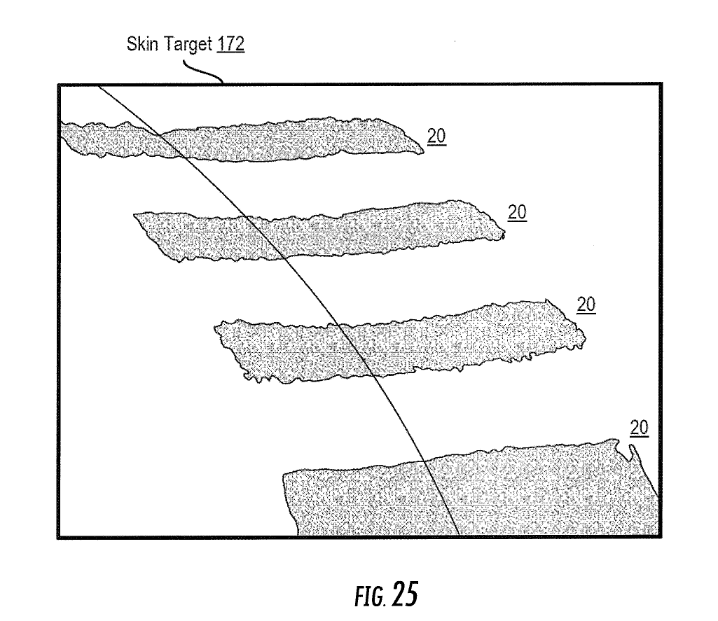

For explanation of a surface of a scanned ear illuminated with laser light, FIG. 23A sets forth a line drawing of a projection onto a surface of an auditory canal of a ring of laser, the ring projected from a conical reflector 132 (FIG. 24A) into a plane which forms a broken ring 134 as the plane of laser light encounters the inner surface of the auditory canal. As the ear probe 106 moves through the auditory canal 202, an image sensor in the scanner captures a sequence 135 of images of the interior of the auditory canal illuminated by rings of projected laser light. Each such image is associated with tracking information gathered by tracking apparatus as illustrated and described with regard to FIG. 18. A combination of such images and associated tracking information is used according to embodiments of the present invention to construct 3D images of a scanned ear.

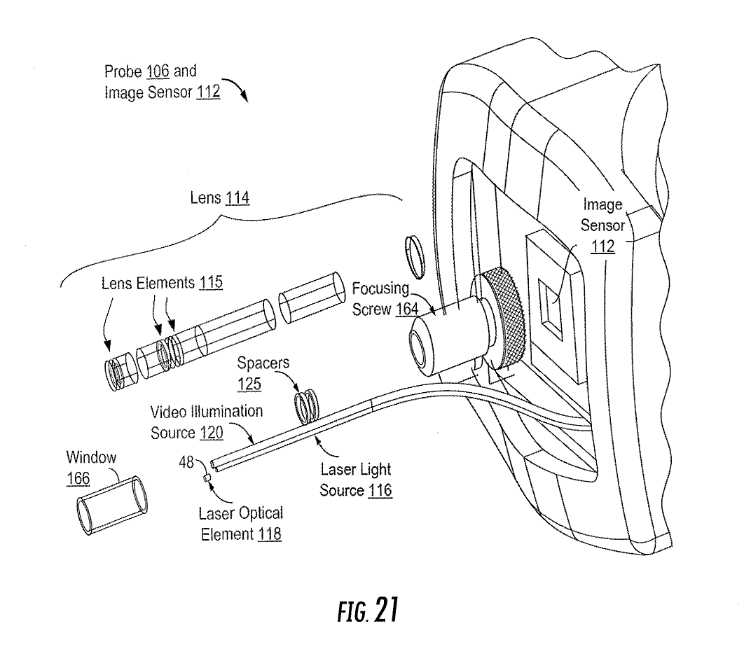

For further explanation of a surface of a scanned ear illuminated with laser light, FIG. 19B sets forth a line drawing of a projection onto surface of a pinna or aurical of a scanned ear of a fan 138 of laser, the fan projected from a diffractive laser lens 136 (FIG. 23A) into a fan shape which illuminates the surface of the pinna, conforming to the surface of the pinna as the fan of laser light encounters the pinna. As an ear probe 106 is moved to scan the pinna, an image sensor in the scanner captures a sequence 137 of images of the surface of the pinna as illuminated by the fan 138 of projected laser light. Each such image is associated with tracking information gathered by tracking apparatus as illustrated and described with regard to FIG. 18. A combination of such images and associated tracking information is used according to embodiments of the present invention to construct 3D images of a scanned ear.

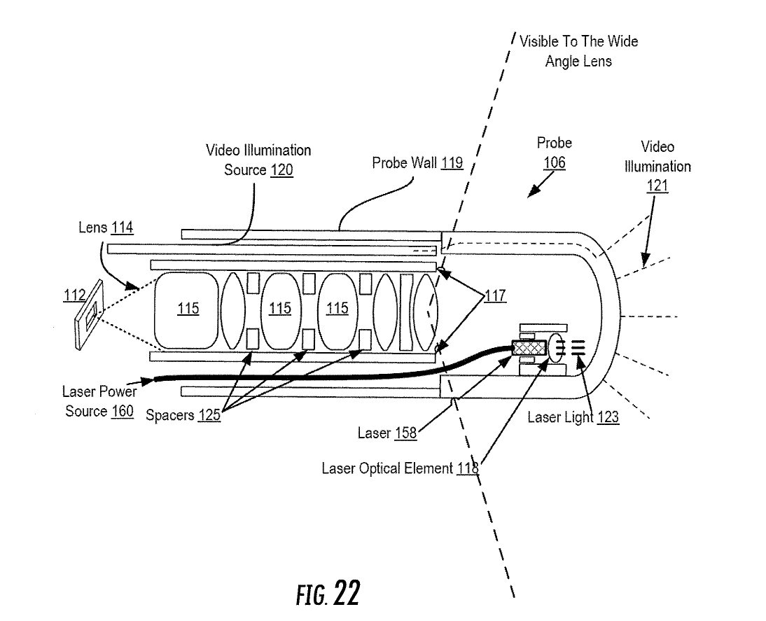

For further explanation of construction of 3D images with a scanner according to embodiments of the present invention, FIG. 20 sets forth a flow chart illustrating an example method of constructing a 3D image of a scanned ear. The method of FIG. 20 includes capturing 302, with an image sensor 112 of a scanner of the kind described above, a sequence 304 of 3D images of surfaces of a scanned ear. The sequence of images is a sequence of 2D images of surfaces of the scanned ear illuminated with laser light as described above. The image sensor includes an array of light-sensitive pixels, and each image 304 is a set of pixel identifiers such as pixel numbers or pixel coordinates with a brightness value for each pixel. The sequence of 2D images is used as described to construct a 3D image.

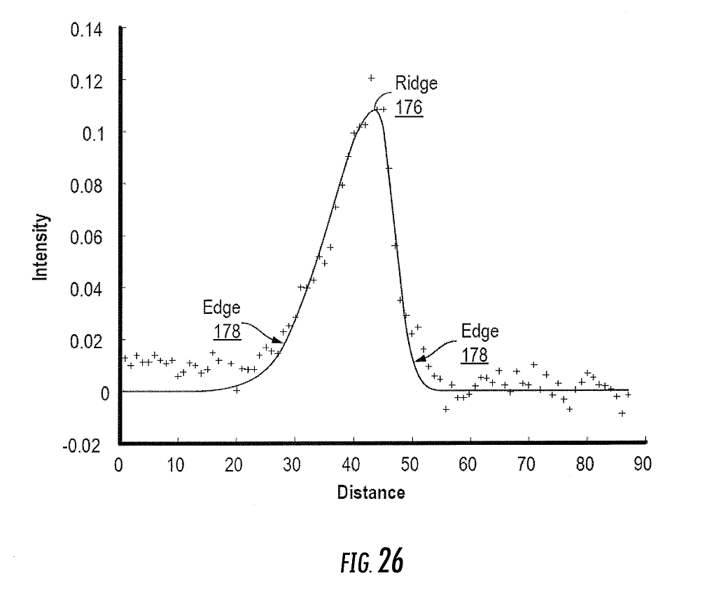

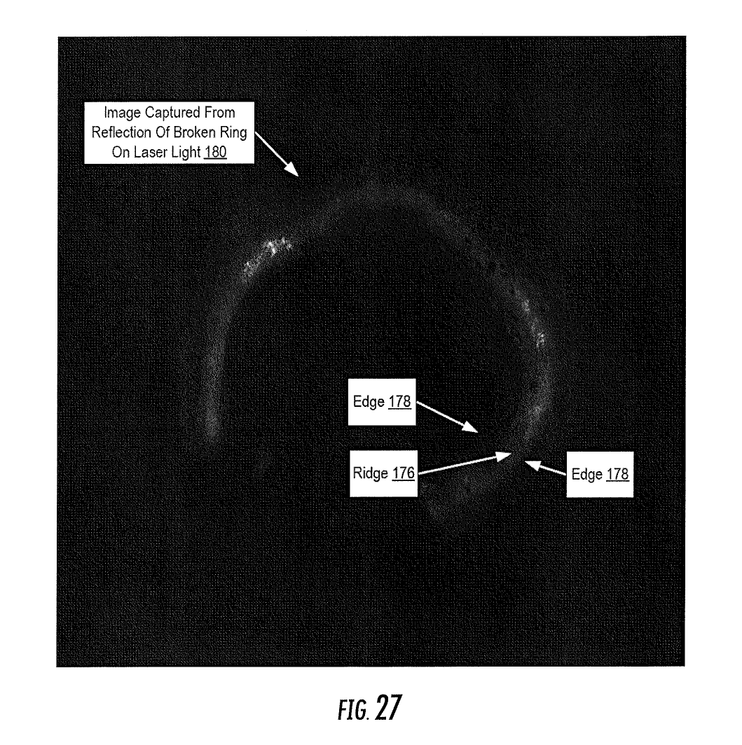

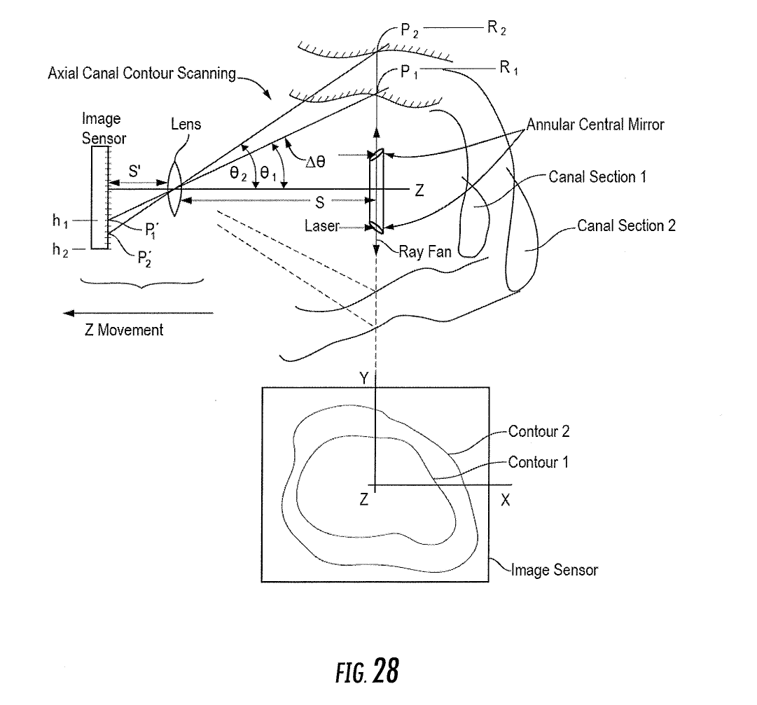

The method of FIG. 20 also includes detecting 306 ridge points 308 for each 2D image. Ridge points for a 2D image make up a set of brightest pixels for the 2D image, a set that is assembled by scanning the pixel brightness values for each 2D image and selecting as ridge points only the brightest pixels. An example of a 2D image is set forth in FIG. 26, illustrating a set of brightest pixels or ridge points 176 that in turn depicts a C-shaped broken ring of laser light reflecting from a surface of an auditory canal of a scanned ear.

The method of FIG. 20 also includes transforming 318 the ridge points to points in scanner space. The transforming 318 in this example is carried out by use of a table of predefined associations 312 between each pixel in the image sensor 112 and corresponding points in scanner space. Each record of table 312 represents an association between a pixel 326 of the image sensor 112 and a point in scanner space 200 (FIG. 18). In the example of table 312, n pixels are identified with numbers, 1, 2, 3, . . . , n−1, n. The pixels of the image sensor can be identified by their x,y coordinates in the image sensor itself, or in other ways as will occur to those of skill in the art. The correspondence between pixels and points in scanner space can be established as described and illustrated below with reference to FIG. 20, triangulation according to equations 2-8. Such triangulation can be carried out by data processor and algorithm for each pixel of each captured frame from the image sensor, although that is computationally burdensome, it is feasible with a fast processor. As a less computationally intense alternative, the triangulation can be carried out once during manufacture or calibration of a scanner according to embodiments of the present invention, with the results stored, for example, in a structure similar to Association table 312. Using such stored associations between pixels and points in scanner space, the process of transforming 310 ridge points to points in scanner space is carried out with table lookups and the like rather than real time triangulations.

The example table 312 includes two columns, one labeled ‘Pixel’ that includes values identifying pixels, and another labeled ‘Coordinates’ that identifies the locations in scanner space that correspond to each pixel. Readers will recognize that in embodiments in which the records in table 312 are sorted as here according to pixel location, then the ‘Pixel’ column actually would not be needed because the position of coordinates in the ‘Coordinates’ columns would automatically index and identify corresponding pixels. In embodiments that omit the ‘Pixel’ columns based on such reasoning, the Associations table 312 is effectively simplified to an array of coordinates. In fact, the data structures of table and array are not limitation of the invention, but instead are only examples of data structures by which can be represented correspondence between pixels and points in scanner space. Readers will recognize that many data structures can be so used, including, for example, C-style structures, multi-dimensional arrays, linked lists, and so on.

The method of FIG. 20 also includes transforming 318 the points 314 in scanner space 200 (FIG. 18) to points 320 in ear space 198 (FIG. 18). This transforming 318 is carried out according to a relationship between an origin 151 (FIG. 18) of a coordinate system defining scanner space 200 (FIG. 18) and an origin 150 (FIG. 18) of another coordinate system defining ear space 198 (FIG. 18). That is, scanner space is both translated and rotated with respect to ear space, and this relationship differs from frame to frame as a scanner is moved in ear space during a scan. The relationship for each frame is expressed as Tensor 1.

$$[ \begin{matrix}{R}_{11} & {R}_{12} & {R}_{13} & {T}_{1} \\ {R}_{21} & {R}_{22} & {R}_{23} & {T}_{2} \\ {R}_{31} & {R}_{32} & {R}_{33} & {T}_{3} \\ 0 & 0 & 0 & 1\end{matrix} ] \qquad Tensor 1$$

The T values in Tensor 1 express the translation of scanner space with respect to ear space, and the R value express the rotation of scanner space with respect to ear space. With these values in Tensor 1, the transformation of points in scanner space to points in ear space is carried out according to Equation 1.

$$[ \begin{matrix}{x}^{′} \\ {y}^{′} \\ {z}^{′} \\ 1\end{matrix} ] ≡ [ \begin{matrix}{R}_{11} & {R}_{12} & {R}_{13} & {T}_{1} \\ {R}_{21} & {R}_{22} & {R}_{23} & {T}_{2} \\ {R}_{31} & {R}_{32} & {R}_{33} & {T}_{3} \\ 0 & 0 & 0 & 1\end{matrix} ] [ \begin{matrix}x \\ y \\ z \\ 1\end{matrix} ] \qquad Equation 1$$

Equation 1 transforms by matrix multiplication with Tensor 1 a vector representing point x,y,z in scanner space into a vector representing point x′,y′,z′ in ear space. The transforming 318 of points in scanner space to points in ear space can be carried out by establishing Tensor 1 for each image scanned from the image sensor and applying Equation 1 to each point 314 in scanner space represented by each pixel in each image.

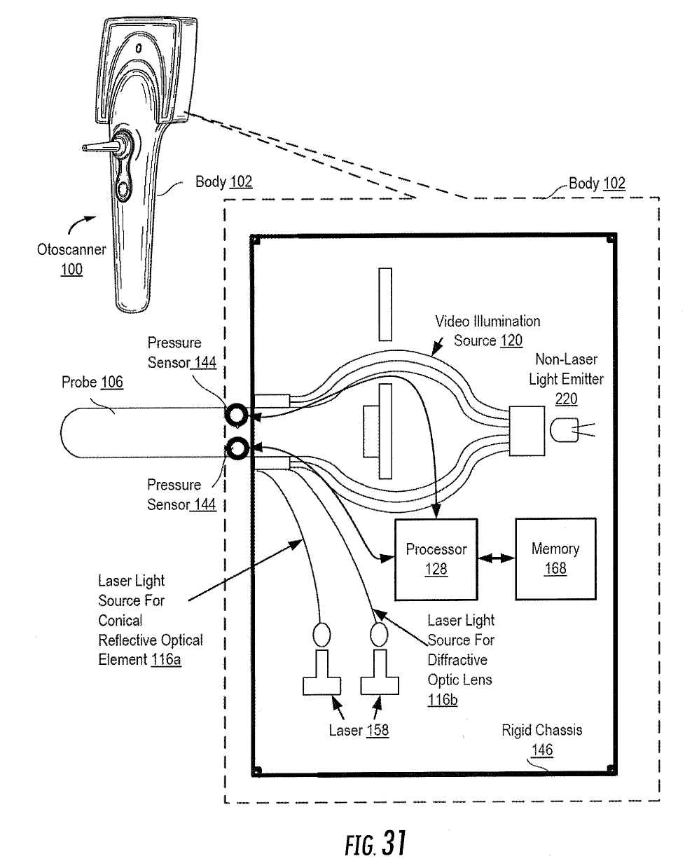

The method of FIG. 20 also includes summing 321 the points in ear space into a 3D image 325 of an ear. The results of such summing are shown schematically in FIG. 29, and an actual 3D image of a scanned ear is set forth in FIG. 30. The image in FIG. 29 was created using the transformed points in ear space as such to display a 3D image. Such a set of points is a mathematical construct. In 3D computer graphics generally, 3D modeling is developing a mathematical representation of a three-dimensional surface of an object (living or inanimate). The products of such processes are called 3D images or 3D models. Such images can be displayed as a two-dimensional image through a process called 3D rendering or used in a computer simulation of physical phenomena. Such an image or model can also be used to create an actual three-dimensional object of a scanned object, such as a scanned ear, using a 3D model as an input to a CAD/CAM process or a 3D printing device.

The method of FIG. 20 also includes determining 324 whether a scan is complete. This determination is carried out by comparing the summed set of points in ear space that now make up a 3D image of the scanned ear for completeness by comparing the 3D image with scanning requirements 322 as specified for a particular, pre-selected class, make, and model of an object to be worn in the ear, an auditory bud, in-ear headphone, hearing aid, or the like. If the scan is incomplete, portions of the 3D image will not meet the scanning requirements as specified for the class, make, and model of the object to be worn in the ear. Often the incomplete portions of the 3D image will appear as holes in the 3D image.

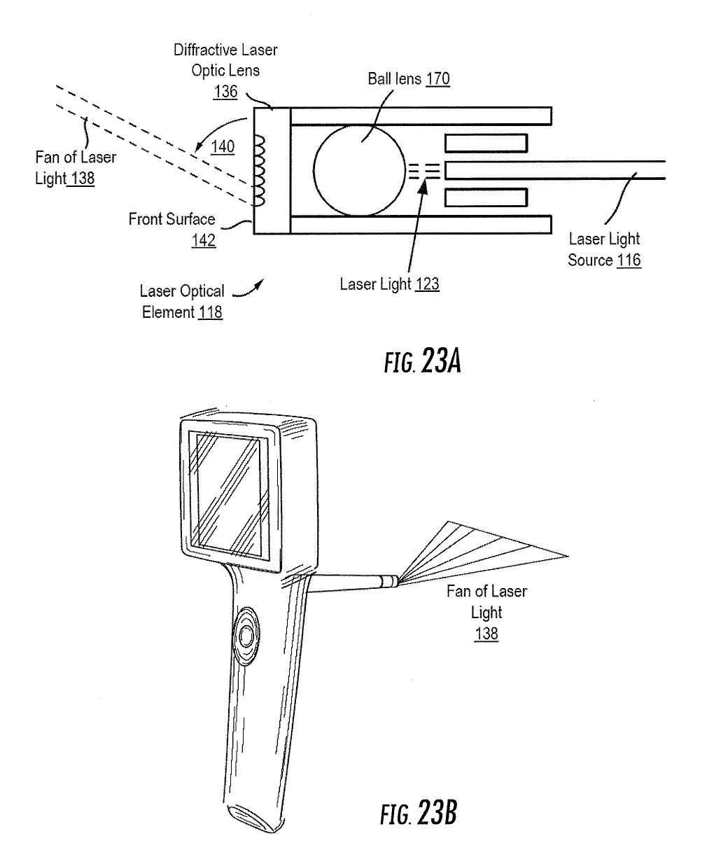

For further explanation, FIG. 21 sets forth a line drawing illustrating additional example features of an ear probe 106 and image sensor 112 of a scanner according to some embodiments. The probe 106 of FIG. 21 has a wide angle lens 114 that includes a number of lens elements 115 and spacers 125. The wide angle lens 114 of FIG. 21 has a sufficient depth of field so that the entire portion of the interior surface of the ear 126 illuminated by laser light is in focus at the image sensor 112. An image of a portion of the ear is said to be in focus if light from object points on the interior of the ear is converged as much as reasonably possible at the image sensor, and out of focus if light is not well converged. Supporting the wide angle lens 114 of FIG. 21 is a focusing screw 164 that when turned adjusts the focus of the wide angle lens 114 for improved accuracy and for compensating for manufacturing tolerances.

The probe 106 of FIG. 21 also includes a laser light source 116 and a laser optical element 118. In the example of FIG. 21 the laser light source 116 is a fiber optic cable carrying laser light from a laser within the body of the scanner to the laser optical element. As mentioned above, in some embodiments of scanners according to the present invention, the laser optical element 118 may include a conical laser reflective optical element. In such embodiments, the lens elements 115 of the wide angle lens 114 of FIG. 21 has sufficient depth of field so that the portion of the interior surface of the ear 126 illuminated by laser light is in focus at the image sensor 112 when the interior surface of the ear is illuminated by a ring of laser light created by use of the conical laser reflective optical element and projected through the transparent side walls of the window 166. In some other embodiments of the present invention, the laser optical element 118 may include a diffractive laser optic lens. In such embodiments, the lens elements 115 of the wide angle lens 114 of FIG. 21 has sufficient depth of field so that the portion of the interior surface of the ear 126 illuminated by laser light is in focus at the image sensor 112 when the interior surface of the ear is illuminated by a fan of laser light created by use of a diffractive laser optic lens and projected through the front of the transparent window 116.

In the example of FIG. 21, the image sensor 112 operates at a video frame rate that is twice a standard video frame rate. By operating at twice a standard video frame rage the image sensor may capture usable video of the scanned ear as well as capture images of the scanned ear for constructing 3D images of the scanned ear. In the example of FIG. 21, therefore, the laser light source 116 is strobed during capture by the image sensor 112 of alternate video frames thereby allowing every other video image to be a 2D image for constructing 3D images. The 2D image for constructing 3D images are captured by the image sensor only when the strobed laser light illuminates the scanned ear. Video frames are captured by the image sensor 112 when only the non-laser video illumination from the video illumination source 120 illuminates the scanned ear.

In the example of FIG. 21, the laser light source 116 of FIG. 21 completely overpowers the video illumination source 120. The video illumination source 12 therefore may remain on such that non-laser video illumination is on during operation of the scanner. Therefore, when the laser light source 116 is strobed, it completely overpowers the video illumination and each time the laser light source illuminates the scanner ear with laser light images captured by the image sensor are 2D images of the scanned ear for construction of a 3D image.

For further explanation, FIG. 22 sets forth a line drawing of an example ear probe 106 of a scanner according to an embodiment. The ear probe 106 of FIG. 22 is similar to the ear probe of FIG. 17A in that it includes a lens 114 with lens elements 115 and spacers 125, a lens tube 117 a video illumination source, a probe wall 119, and a laser optical element 118. The field of view of the illustrated embodiment, shown by dotted lines, is approximately 150 degrees, although the light pattern 123 may extend laterally out at right angles to the optical axis of the wide angle lens 114. Angles up to 180 degrees are possible but wider angles can be increasingly difficult to minimize distortion. The ear probe 106 of FIG. 22 differs from the ear probe of FIG. 17A in that the laser light source of the ear probe of FIG. 18 is a laser 158 mounted in the probe 106 itself. In the example of FIG. 22 the laser 158 is mounted in the probe and power to the laser is proved by a laser power source 160 delivering power from within the scanner body. In some embodiments, the laser may be a mounted on a bare die allowing the laser to be placed directly on a printed circuit board in the ear probe.

As mentioned above, scanners according to embodiments of the present invention may be configured to project a ring of laser light radially from the tip of the distal end of the ear probe, project a fan of laser light forward from the tip of the distal end of the ear probe, or configured to project other shapes of laser light as will occur to those of skill in the art. For further explanation, therefore, FIGS. 23A and 23B set forth line drawings of an optical element 118 useful in scanners according to embodiments of the present invention and a resultant fan of laser light 138 projected from an ear probe having such an optical element. The laser optical element 118 of FIG. 23A comprises a diffractive laser optic lens 136. In the example of FIG. 23A, the laser light source 116 and the diffractive laser optic lens 136 are configured so that when illuminated by the laser light source 116 the diffractive laser optic lens 136 projects upon an interior surface of the ear a fan 138 of laser light at a predetermined angle 140 with respect to a front surface 142 of the diffractive laser optic lens 136. In the example of FIGS. 23A and 23B, laser light from the source of laser light 116 is focused by a ball lens 170 on the diffractive laser optic lens 136. The diffractive laser optic lens 136 diffracts the laser light into a fan 138 of laser light. The diffractive laser optic lens 136 is manufactured to diffract the laser light at a predetermined angle 140 from its front surface 142 into a fan of laser light 138 as illustrated in FIGS. 23A and 23B.

As mentioned above, scanners according to embodiments of the present invention may be configured to project a ring of laser light radially from the tip of the distal end of the ear probe. For further explanation, therefore, FIGS. 24A and 20B set forth line drawings of an optical element 118 useful in scanners according to embodiments of the present invention and a resultant ring of laser light 134 projected from an ear probe having such an optical element. The laser optical element 118 of FIG. 24A includes a conical laser-reflective optical element 132. In the example of FIG. 24A the laser light source 116 and the conical laser-reflecting optical element 132 are configured so that the conical laser-reflecting optical element 132, when illuminated by the laser light source 116, projects a broken ring 134 of laser light upon an interior surface of the ear when the ear probe is positioned in the ear. In the example of FIGS. 22A and 22B, laser light from the laser light source 116 is focused by a ball lens 170 onto the conical laser reflective optical element 132. The conical laser reflective optical element 132 reflects the laser light into a ring of laser light 134 as illustrated in FIGS. 24A and 24B.

In the examples of FIGS. 24A and 24B the ring of laser light is broken because the conical laser reflective optical element 132 is mounted in a fashion that blocks a portion of the laser light reflected by the optical element. In alternate embodiments, however, the ring of laser light reflected by the conical laser reflective optical element 132 is unbroken as will occur to those of skill in the art.

Referring to FIG. 25, a skin target is shown with partial lateral portions 20 of rings of laser light projected thereon for the purpose of determining how the laser light will project upon skin and its location be marked. A perpendicular section of one of the lateral portions, as shown in FIG. 26, illustrates the fact that the reflected laser light intensity (y-axis) varies in a bell-curve shape with the thickness (x-axis) of the section. Thus, the partial lateral portion 20 may include an edge 22 of the light pattern as well as a ridge 24 of the light pattern. These landmarks may be used to determine the position of the lateral portion 20 in a coordinate system defining an ear space. For example, one of the aforementioned landmarks could be found (such as by a ridge detecting function of a data processor) or an inside edge of the lateral portion or an outside edge of the lateral portion. Or, an average of the inside and outside portions may be used.

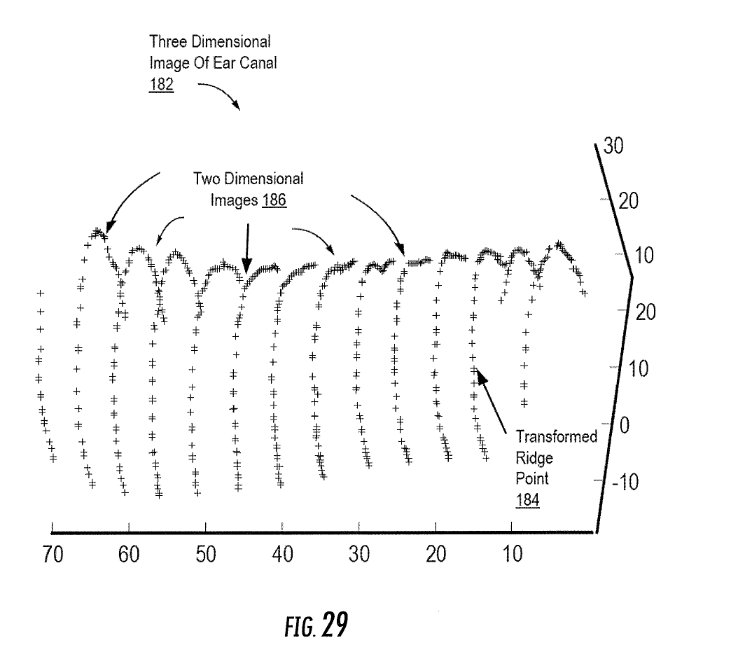

For further explanation, FIG. 27 sets forth an image captured from reflections of laser light reflected from a conical laser reflective optical element 132 radially from the tip of the ear probe of a scanner according to embodiments of the present invention. The captured image of FIG. 27 forms a c-shaped broken ring of pixels of highest intensity. Along the outside and inside of the broken ring 180 are pixels of intensity defining an edge as mentioned above. In between the edges 178 of the broken ring are pixels of higher intensity that define a ridge. The ridge 176 is a collection of ridge points that comprise a set of brightest pixels for the captured 2D image.

Constructing a 3D image of the interior of a scanned ear according to embodiments of the present invention for a sequence of 2D images of the ear such as the image of FIG. 27 includes detecting ridge points for each 2D image. Detecting ridge points in the example of FIG. 27 includes identifying a set of brightest pixels for the 2D image. In the example of FIG. 27, ridge points are detected as a set of brightest pixels along the ridge 176 of the image 180. Detecting ridge points may be carried out by scanning across all pixels in a row on the image sensor and identifying a pixel whose intensity value is greater than the intensity values of pixels on each side. Alternatively, detecting a ridge point may be carried out by identifying range of pixels whose average intensity values are greater than the intensity values of a range of pixels on each side and then selecting one of the pixels in the range of pixels with greater average intensity values. As a further alternative, detecting ridge points can be carried out by taking the brightest pixels from a purposely blurred representation of an image, a technique in which the pixels so selected generally may not be the absolute brightest. An even further alternative way of detecting ridge points is to bisect the full-width half maximum span of a ridge at numerous cross sections along the ridge. Readers will recognize from this description that constructing a 3D image in this example is carried out with some kind of ridge detection. In addition to ridge detection, however, such construction can also be carried out using edge detection, circle detection, shape detection, snakes detection, deconstruction techniques, and in other ways as may occur to those of skill in the art.

Constructing a 3D image of the interior of a scanned ear according to embodiments of the present invention for a sequence of 2D images also includes transforming, in dependence upon a predefined association between each pixel in the image sensor and corresponding points in scanner space, the ridge points to points in scanner space as described with reference to FIG. 27 and transforming, in dependence upon a relationship between an origin of a coordinate system defining scanner space and an origin of another coordinate system defining ear space, the points in scanner space to points in ear space as described with reference to FIG. 29.

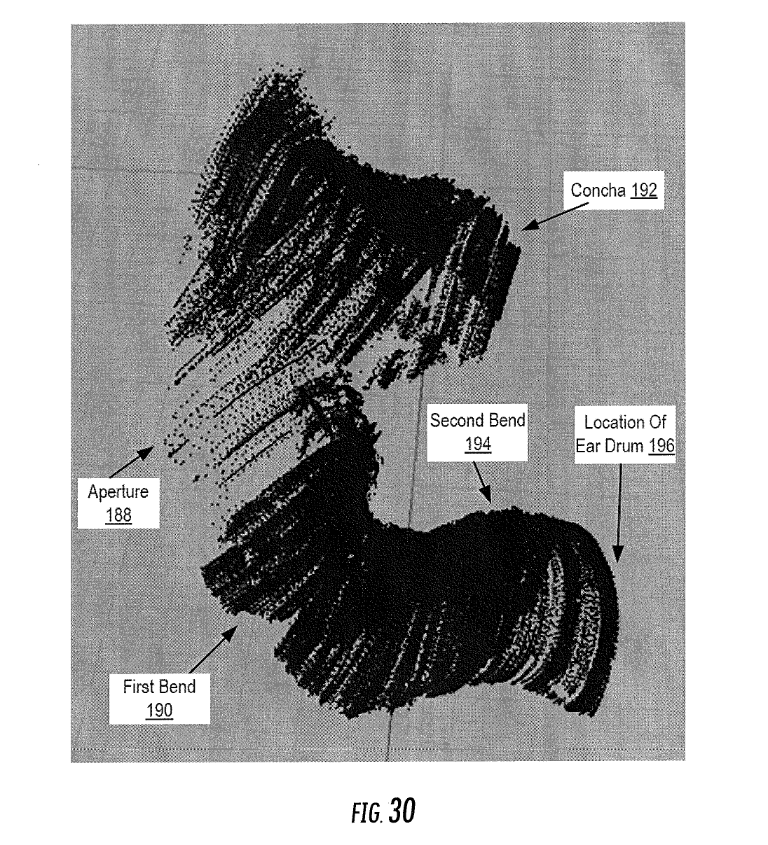

For further explanation, FIG. 28 sets forth a line drawing schematically illustrating transforming, in dependence upon a predefined association between each pixel in the image sensor and corresponding points in scanner space, the ridge points to points in scanner space. FIG. 28 schematically shows an embodiment for calculation of the radial distance of the lateral portion from the optical axis of the probe as implemented by a data processor. The position can be determined by triangulation, as shown in equations 2-8.

$$\begin{matrix}\frac{h}{{S}^{′}} ≡ \frac{R}{S} & Equation 2 \\ R = \frac{hS}{{S}^{′}} & Equation 3 \\ \frac{{S}^{′}}{S} = M & Equation 4 \\ R = \frac{h}{M} & Equation 5 \\ Δ R = \frac{\text{?}}{M} & Equation 6 \\ {θ}_{min} = {Tan}^{- 1} ( \frac{{R}_{min}}{S} ) & Equation 7 \\ {θ}_{max} = {Tan}^{- 1} ( \frac{{R}_{max}}{S} ) \\ \text{?} \text{indicates text missing or illegible when filed} & Equation 8\end{matrix}$$

In the example of FIG. 28 and in equations 2-8, scanner space is oriented so that its Z axis is centered and fixed as the central axis of an ear probe, looking end-on into the probe, here also referred to as the imaging axis. In this example, therefore, the ratio of the distance R from the imaging axis of a laser-illuminated point to the distance S between the laser plane and the lens is equal to that of the distance h from the center of the image sensor to the distance S′ between the image sensor surface and the lens. Magnification M is the ratio of S′ and S. When the distances S and S′ between the lens and laser plane, and lens to image sensor are known, equations 2-8 can reconstruct the geometry of illuminated points in scanner space. These equations also denote that for a focal surface such as a plane, there is a 1:1 mapping of points in scanner space to pixel locations on the image sensor.

The image sensor 112 may be implemented in complementary-symmetry metallic-oxide-semiconductor (‘CMOS’) sensor, as a charge-coupled device (‘CCD’), or with other sensing technology as may occur to those of skill in the art. A CMOS sensor can be operated in a snapshot readout mode or with a rolling shutter when the scan along the Z-axis is incremented or stepped synchronously to effect a readout of a complete frame. Similar incrementing or stepping may be used for a CCD operated with interlacing scans of image frames.

Constructing a 3D image of the interior of a scanned ear according to embodiments of the present invention also often includes transforming, in dependence upon a relationship between an origin of a coordinate system defining scanner space and an origin of another coordinate system defining ear space, the points in scanner space to points in ear space. For further explanation, therefore, FIG. 29 sets forth a line drawing illustrating an exemplary three-dimensional image (182) of an ear canal constructed from a sequence of 2D images by a data processor. In the example of FIG. 29, each of the 2D images (186) includes a set of transformed ridge points. The transformed ridge points are the result of transforming, in dependence upon a relationship between an origin of a coordinate system defining scanner space and an origin of another coordinate system defining ear space, the points in scanner space to points in ear space as described with reference to FIG. 29. Transforming, in dependence upon a relationship between an origin of a coordinate system defining scanner space and an origin of another coordinate system defining ear space, the points in scanner space to points in ear space may be carried out by as described and illustrated above with reference to FIG. 20.

For further explanation, FIG. 30 sets forth a 3D image of a scanned ear created by use of a scanner and 3D imaging according to embodiments of the present invention. The 3D image of FIG. 30 includes a 3D depiction of the concha 192, the aperture 188 of the ear, the first bend 190 of the ear canal, the second bend of the ear canal and the location of the ear drum 196. The 3D image of FIG. 30 may be used by a manufacturer to provide a custom fit orthotic.

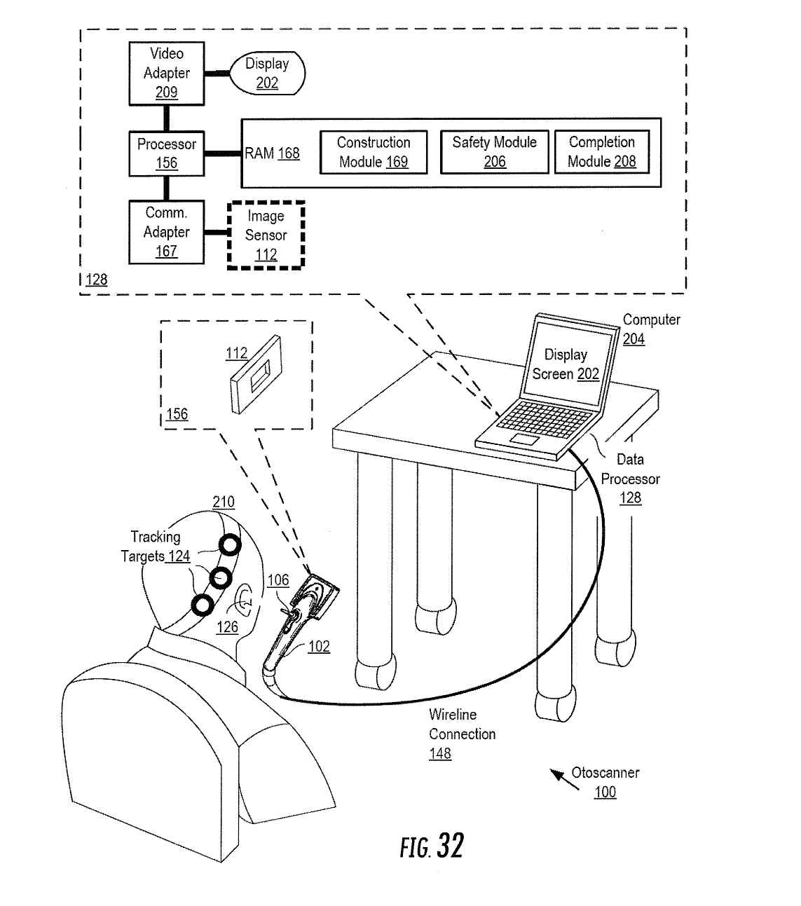

The density of portions of the skin making up the ear varies from person to person. The density of portions of the skin making up the ear also varies across the portions of the ear. That is, some people have ears with skin that is more compliant in certain areas of the ear than others. The compliance of the skin of an ear is a factor in determining whether a custom orthotic in the ear is comfortable to its wearer while still providing a proper fit within the ear. Compliance information may be provided to a manufacturer for use making a comfortable and well fitting hearing aid, mold, or other object worn in the ear. For further explanation, therefore, FIG. 31 sets forth a line drawing of a scanner capable of detecting the force with which the ear probe is pressed against a surface of the scanned ear for use in calculating a compliance value as an aid to a manufacturer in making comfortable and well fitting objects worn in the ear. The scanner 100 of FIG. 31 is similar to the scanner of FIGS. 17 and 18 in that the scanner has a body 102, an ear probe 106, video illumination source 120 carrying video illumination from a non-laser light emitter 220, a laser light source for a conical reflective optical element 116a carrying laser light from a laser 158a in the body 102 of the scanner 100, a laser light source for a diffractive optical lens 116b carrying light from a laser 158b in the body 102 of the scanner 100 and so on.

The scanner 100 of FIG. 31 differs from the scanner of FIGS. 17 and 18 in that the scanner body 102 has mounted within it pressure sensors 144 operably coupled to the ear probe 106. In the example of FIG. 31, the pressure sensors 144 are coupled for data communications to the data processor 128 and pressure sensors detect the force with which the ear probe 106 is pressed against a surface of the scanned ear. In some embodiments, the probe is implemented as entirely rigid when scanning. In other embodiments, the probe is implemented as somewhat moveable against pressure sensors for compliance measurements. And some embodiments implement a probe that is alternately both rigid and moveable, providing a locking mechanism that maintains the probe as rigid for optical scanning and allows the probe to move against a pressure sensor when unlocked for ascertaining a compliance value.

The scanner 100 is also configured to track positions of the ear probe inferred from reflections of tracking illumination sensed by the tracking illumination sensors 108. The tracked positions are used to identifying the displacement through which the ear probe 106 moves when pressed against the surface of the scanned ear. The data processor 128 of FIG. 31 is further configured so that it functions by calculating a compliance value in dependence upon the detected force and the tracked displacement. The compliance value may be implemented as a single value or range of values dependent upon the detected force and the identified displacement when the probe is pressed against the surface of the scanned ear.

To facilitate the detection of the force when the probe is pressed against the surface of the scanned ear, the scanner body 102 has mounted within it pressure sensors 144 operably coupled to the ear probe 106. The tracking sensors 108, the image sensor 112, the probe 106 and lens of the scanner 100 of FIG. 31 are all mounted on a rigid chassis 146 that is configured to float within the scanner body 102. The pressure sensors 144 are mounted within the scanner 100 between the rigid chassis 146 and the scanner body 102. The rigid chassis 146 is floated in the body 102 of the scanner 100 in that the rigid chassis 146 may move relative to the body 102 of the scanner 100 when the probe 106 is pressed against the surface of the ear.

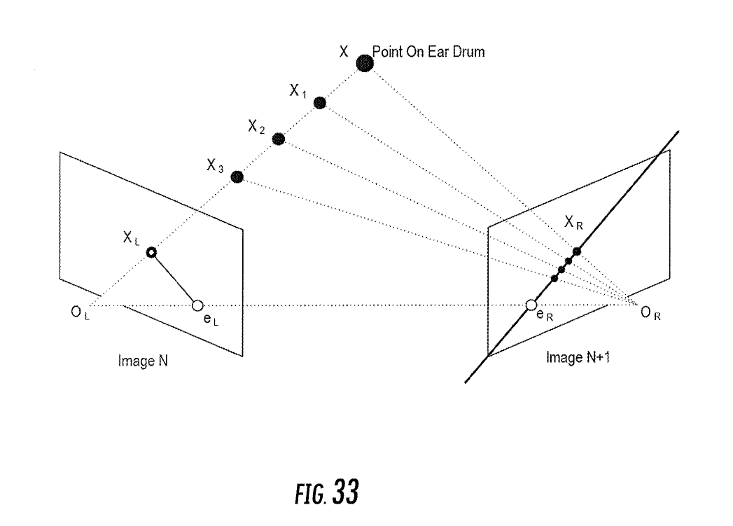

In the example scanners described above, the functionality of the scanner is described as residing within the body of the scanner. In some embodiments, a scanner may be configured with a wireline connection to a data processor 128 in a computer 202 available to an operator of the scanner. For further explanation, therefore, FIG. 32 sets forth a further example scanner according to embodiments of the present invention that includes a scanner body 102 with a wireline connection 148 to a data processor 128 implemented in a computer 204. In the example of FIG. 32 the elements of the scanner are distributed between the scanner body 102 and the computer 204. In the example of FIG. 32, the tracking targets 124 are fixed to a headband worn by the person whose ear 126 is being scanned.

The data processor 128 in the computer 204 of FIG. 32 includes at least one computer processor 156 or ‘CPU’ as well as random access memory 168 (‘RAM’) which is connected through a high speed memory bus and bus adapter to processor the 156 and to other components of the data processor 128. The data processor 128 of FIG. 32 also includes a communications adapter 167 for data communications with other computers and with the scanner body 102 and for data communications with a data communications network. Such data communications may be carried out serially through RS-232 connections, through external buses such as a Universal Serial Bus (‘USB’), through data communications networks such as IP data communications networks, and in other ways as will occur to those of skill in the art. Communications adapters implement the hardware level of data communications through which one computer sends data communications to another computer, directly or through a data communications network. The example data processor FIG. 32 includes a video adapter (209), which is an example of an I/O adapter specially designed for graphic output to a display device (202) such as a display screen or computer monitor.

In the example of FIG. 32, the image sensor 112 is illustrated in callout 156 as residing within the scanner body as well as being illustrated in callout 128 as residing in the data processor. An image sensor useful in embodiments of the present invention illustrated in FIG. 32 may reside in either location, or as illustrated in callout 156 or in the computer 202 itself.

In the example of FIG. 32, a display screen 202 on the computer 204 may display images of the scanned ear scanned ear illuminated only by non-laser video illumination 120. The display screen 202 on the computer 113 may also display 3D images of the scanned ear constructed in dependence upon a sequence of images captured by the image sensor as the probe is moved in the scanned ear. In such examples images captured by an image sensor 112.

Stored in RAM 168 in the data processor 128 of FIG. 32 is a construction module. A module of computer program instructions for constructing 3D images of the scanned ear in dependence upon a sequence of images captured by the image sensor 112 as the probe is moved in the scanned ear. The construction module 169 is further configured to determine the position of the probe 106 in ear space when the probe is positioned at the aperture of the auditory canal of the scanned ear 126 and setting the position of the probe at the aperture of the auditory canal of the scanned ear as the origin of the coordinate system defining ear space.

There is a danger to an ear being scanned if a probe or other object is inserted too deeply in the ear. For example, an ear drum may be damaged if it comes into contact with a probe. Also stored in RAM 206, therefore, is a safety module 206, a module of computer program instructions for safety of use of the scanner 100 of FIG. 32. The safety module 206 of FIG. 32 has a database of previously recorded statistics describing typical ear sizes according to human demographics such as height weight, age and other statistics of the humans. The safety module 206 also has currently recorded demographic information regarding a person whose ear is being scanned. The safety module infers, from a tracked position of the ear probe 106, previously recorded statistics describing typical ear sizes according to human demographics, and currently recorded demographic information regarding a person whose ear is scanned, the actual present position of the ear probe in relation to at least one part of the scanned ear. The safety module is configured to provide a warning when the probe moves within a predefined distance from the part of the scanned ear. Such a warning may be implemented as a sound emitted from the scanner 100, a warning icon on a display screen of the scanner 100 or computer, or any other warning that will occur to those of skill in the art.

Those of skill in the art will recognize that the ear is flexible and the shape of the ear changes when the mouth of the person being scanned is open and when it is closed. To facilitate manufacturing an orthotic worn in the ear in the example of FIG. 32, an operator scans the ear with the scanner of FIG. 32 with the mouth open and then with the mouth closed. 3D images of the ear constructed when the mouth is open and also when the mouth is closed may then be used to manufacture a hearing aid, mold, or other object worn in the ear that is comfortable to the wearer when the wearer's mouth is open and when it is closed. The construction module 169 of the data processor 128 of FIG. 32 is therefore configured to construct the 3D image of the scanned ear by constructing the 3D image in dependence upon a sequence of images captured by the image sensor as the probe is moved in the scanned ear with mouth open. The construction module 169 of the data processor 128 of FIG. 32 is also configured to construct the 3D image of the scanned ear by constructing the 3D image in dependence upon a sequence of images captured by the image sensor as the probe is moved in the scanned ear with mouth closed.

In-ear device 50 shown in FIGS. 8-10 is a non-limiting example of a customized in-ear orthotic designed from a 3D image constructed from optical scanning of the ear canal as described above.

According to some embodiments, the in-ear device can be customized based on the shape of the subject's ear canal when the subject's jaw is in the therapeutic or optimal position. The therapeutic or optimal position of the jaw can be determined using any desired conventional method. For example, some believe that the therapeutic or optimal position of the jaw is when the jaw is in a forward position. One skilled in the art will appreciate that the therapeutic or optimal jaw position may be determined using any one of a number of known methods. For example, the therapeutic or optimal position may be determined by indexing of the jaw. This position may also be determined by aligning the lower jaw and the upper jaw in a predetermined manner such as at their midpoints. The position may alternatively be determined using the swallow technique, selecting the position phonetically (when the jaw is positioned as certain sounds are made), or by arbitrarily selecting what appears to be the therapeutic or optimal position based on visual inspection. Once the jaw is in this therapeutic or optimal position, the position can be indexed with wax or bite registration material. This wax or bite registration can be used to maintain the jaw in its therapeutic or optimal position. While the jaw is maintained in this therapeutic or optimal position, the ear canal may be scanned as described above and a 3D image of the ear canal when the jaw is in the therapeutic or optimal position may be generated. In this way, the device is custom designed so that it conforms to the ear canal when the jaw is in the therapeutic or optimal position and so that it deforms the ear canal when the jaw moves out of the therapeutic or optimal position and/or moves past a predetermined threshold. As discussed above, the rigidity or softness of the device can be varied to meet the particular needs of the subject.

In some embodiments, the in-ear device may be customized using scans of the outside of a subject's jaw, either alone or in combination with scans of the subject's ear canal. In addition to scanning, parameters may also optionally be used to customize the in-ear device to the particular subject. For example, the subject's facial type, height, gender, age, demographics, weight, occupation, and other demographic information can be used to help customize the in-ear device. In some cases, the subject's stage in what is known as the Piper classification system for TMD or other parameters or TMD are used to customize the in-ear device. Etiological or pathophysiological parameters or other information from the study of information sciences may be used to customize the in-ear device. Any or all of these various parameters, along with feedback provided by the subject, may be used in a feedback loop to further customize the in-ear device.

In embodiments where the device is customized to the particular subject's ear canal based on the configuration of the ear canal when the subject's jaw is in the therapeutic or optimal position, the device will substantially conform to the subject's ear canal when the subject's jaw is in the therapeutic or optimal position. In this way, the subject will not receive any sensory indications associated with the in-ear device when the subject's jaw is in the therapeutic or optimal position. When the jaw goes beyond the therapeutic or optimal position by a certain predetermined amount (for example, when the subject begins to clench/grind his teeth or closes his jaw beyond the therapeutic or optimal position), the device provides a sensory indication to the subject as described above. In particular, in cases where the subject's ear canal decreases in cross-sectional area when the jaw is closed, the in-ear device will no longer substantially conform to the ear canal when the jaw is closed, causing the in-ear device to exert force on the ear canal when the jaw is clenched or the teeth are grinding (and in some embodiments, to substantially deform the subject's ear canal) and provide a sensory indication to the subject that he should alter movement or position of his jaw to avoid or reduce TMJ-related symptoms.

Also disclosed is a method of scanning the jaw in its therapeutic or optimal position, its closed position, its open position, or any combination thereof to track how the dimensions of that particular subject's ear canal changes. These scans can then be used to determine the positioning of one or more protrusions as described above, including the location of that particular subject's first and second bends. Moreover, if the scans indicate that the cross-sectional area of the subject's ear canal decreases when the jaw is closed and/or open, it might be determined that passive detection as described above is sufficient. On the other hand, if the scans indicate that the cross-sectional area of the subject's ear canal does not decrease when the jaw is closed and/or open, it might be determined that active detection in form of accelerometer, voltage sensor, or other suitable sensor should be incorporated into the in-ear device. Essentially, 3D scanning of the ear can be used to determine the appropriate in-ear device solution for the subject, including the dimensions and/or overall shape of the device and whether to include active indicators in addition to passive indicators.

As described above, tissue hardness and elasticity, ear canal translation, ear canal cross-sectional area change, and subject-specific pain threshold are all input specifications that can be used to create a custom-designed orthotic for the treatment of TMD from the ear canal. In some cases, 3D scans coupled with post-processing allow for relative position and volume analysis. In addition, mechanical factors also can be analyzed to create a custom in-ear device. For example, output parameters such as protrusion radius, relative position, angle, durometer, and wall thickness depend on movements of the mandibular condyle and can affect canal dynamics. As such, 3D scans may not able to completely detect movement of the mandibular condyle since tissue hardness and elasticity attenuates visual motion inside the canal. Moreover, pressure needed for proprioceptive feedback differs from subject to subject, along with tissue hardness and elasticity and ear canal dynamics, and a device that creates unnecessary pain should be avoided. Because sensation and pain are subjective, these factors can be considered individually during the creation of a custom orthotic. To help account for these various factors, a measurement device may be used in conjunction with the methods described above to help design a custom in-ear device. In one embodiment, the device includes a distal end that extends bilaterally and includes an indicator that measures the depth from the ear canal aperture, diameter of the ear canal, and/or angle of application. In some embodiments, the device includes a tension adjuster to determine hardness and elasticity of the tissue, which may help determine the optimum parameters of sensation or pain needed for the orthotic. In some embodiments, the measurement tool may include electrical and computing components such as force sensors, orientation sensors, and interface devices.

It should also be understood that the subject matter described herein may be incorporated into any suitable in-ear device such as hearing aids, ear buds, hearing protection devices, and so forth.

Method of Treating or Preventing One or More TMT-Related Symptoms

Disclosed is a method of treating TMD in a subject by providing the described device to the ear canal of the subject. Optionally, the device is provided to the ear canal during the day when the subject is awake and a mouth guard is provided at night when the subject is asleep and not as receptive to the signals provided by the one or more proprioceptive features.

Also disclosed is a method of treating one or more symptoms of TMD in a subject by creating a customized in-ear device as described above to influence the positioning of the jaw. In particular, the in-ear device can be used to help keep the upper and lower teeth separated so the jaw can move without occlusal (dental) interferences. Over time, the custom in-ear device can be replaced with a new in-ear device that is customized based on the adjusted position and/or movement of the jaw. Over time, the iterative in-ear devices can help influence the movement of the jaw back into its therapeutic or optimal position by accommodating changes in the jaw's position. Although the TMJ disc itself might not reposition into its original location, the use of the in-ear devices can be used to encourage remodeling or even pseudodisc formation to prevent or reduce TMJ-related pain.

Kits

Further provided is a method of treating TMD in a subject wherein the customized in-ear device is modified over time to provide a series of devices, where each device in the series is customized to the subject.

Specifically a kit comprising multiple pairs of in-ear devices may be selectively configured for insertion in the subject's ear canal, where each pair of the in-ear devices is designed to provide progressive adjustment of the temporomandibular joint disorder of the subject.

The foregoing is provided for purposes of illustrating, explaining, and describing embodiments of the present invention. Further modifications and adaptations to these embodiments will be apparent to those skilled in the art and may be made without departing from the scope or spirit of the invention. Different arrangements of the components depicted in the drawings or described above, as well as components and steps not shown or described are possible. Similarly, some features and subcombinations are useful and may be employed without reference to other features and subcombinations. Embodiments of the invention have been described for illustrative and not restrictive purposes, and alternative embodiments will become apparent to readers of this patent. Accordingly, the present invention is not limited to the embodiments described above or depicted in the drawings, and various embodiments and modifications can be made without departing from the scope of the claims below.

We claim:

1. A custom in-ear orthotic comprising:
a surface comprising at least one feature that is customized to a subject's ear canal, wherein the at least one feature provides a sensory indication to the subject when the subject's jaw moves past a predetermined limit in range of motion, and
wherein the in-ear orthotic is customized to the subject's ear canal by optically scanning the subject's ear canal, generating a three-dimensional image of the scanned ear canal and modeling the in-ear orthotic from the generated three dimensional image.

2. The custom in-ear orthotic of claim 1, wherein the at least one feature comprises a protrusion that projects from the surface of the device at an angle that is selected based on the generated three-dimensional image.

3. The custom in-ear orthotic of claim 1, wherein the at least one feature comprises a protrusion that projects from the surface of the device at an angle that is selected based on demographics of the subject.

4. The custom in-ear orthotic of claim 2, wherein the protrusion is configured to align with either a first bend or a second bend of the subject's ear canal.

5. The custom in-ear orthotic of claim 1, wherein the at least one feature is compressible.

6. The custom in-ear orthotic of claim 1, wherein the predetermined limit corresponds to a jaw position associated with clenching the jaw or grinding teeth.

7. The custom in-ear orthotic of claim 1, further comprising at least one sensor for monitoring movement of the subject's jaw and wherein the sensor generates a signal when the subject's jaw moves past the predetermined limit.

8. The custom in-ear orthotic of claim 6, wherein the at least one sensor comprises at least one force sensor that senses force on the subject's ear canal associated with the movement of the subject's jaw.

9. The custom in-ear orthotic of claim 6, wherein the at least one sensor comprises at least one accelerometer that detects acceleration of the subject's jaw.

10. The custom in-ear orthotic of claim 6, wherein the at least one sensor comprises at least one voltage sensor that detects voltage across the subject's muscles or nerves.

11. The custom in-ear orthotic of claim 6, wherein the signal is an auditory signal or a vibration.

12. The custom in-ear orthotic of claim 1, wherein the orthotic comprises a generally C-shaped sound channel.

13. A device adapted to be inserted into an ear canal having a bend for treating discomfort in a joint between a mandible and a corresponding temporal bone, the joint having a disc located between the mandible and the temporal bone and associated musculature, the device having a generally cylindrical core with an exterior surface shaped to substantially conform to a contour of the portion of the ear canal which extends approximately between the entrance to the ear canal and the bend, the device, when inserted, adapted reduce discomfort in the joint,
wherein the device is customized to the ear canal by optically scanning the ear canal to determine a geometry of the ear canal, generating a three-dimensional model of the ear canal using the determined geometry of the ear canal, and modeling the device based on the generated three-dimensional model.

14. A system for reducing one or more symptoms associated with temporomandibular joint disorder comprising:
(1) a scanner comprising:
a scanner body, the body comprising a hand grip, the body having mounted upon it an ear probe, a tracking illumination emitter, a plurality of tracking illumination sensors, and a display screen, the scanner body having mounted within it an image sensor;
the ear probe comprising a wide-angle lens optically coupled to the image sensor, a laser light source, a laser optical element, and a source of non-laser video illumination;
the plurality of tracking illumination sensors disposed upon the scanner body so as to sense reflections of tracking illumination emitted from the tracking illumination emitter and reflected from tracking targets installed at positions that are fixed relative to an ear canal;
the display screen coupled for data communications to the image sensor, the display screen displaying images of the ear canal, the display screen positioned on the scanner body in relation to the ear probe so that, when the ear probe is positioned for scanning, both the display screen and the ear probe are visible to an operator operating the scanner; and
the image sensor coupled for data communications to a data processor, with the data processor configured so that it functions by constructing, in dependence upon a sequence of images captured when the scanned ear is illuminated by laser light and tracked positions of the ear probe inferred from reflections of tracking illumination sensed by the tracking illumination sensors, a 3D image of the interior of the ear canal; and
(2) a device that is modeled from the constructed 3D image, wherein the device is adapted to be inserted into the ear canal, the ear canal having a bend for treating discomfort in a joint between a mandible and a corresponding temporal bone, the joint having a disc located between the mandible and the temporal bone and associated musculature, the device having a generally cylindrical core with an exterior surface shaped to substantially conform to a contour of the portion of the ear canal which extends approximately between the entrance to the ear canal and the bend, the device, when inserted, adapted to reduce discomfort in the joint.

15. A system for reducing one or more symptoms associated with temporomandibular joint disorder in a subject comprising:
(1) a scanner comprising:
a scanner body, the body comprising a hand grip, the body having mounted upon it an ear probe, a tracking illumination emitter, a plurality of tracking illumination sensors, and a display screen, the scanner body having mounted within it an image sensor;
the ear probe comprising a wide-angle lens optically coupled to the image sensor, a laser light source, a laser optical element, and a source of non-laser video illumination;
the plurality of tracking illumination sensors disposed upon the scanner body so as to sense reflections of tracking illumination emitted from the tracking illumination emitter and reflected from tracking targets installed at positions that are fixed relative to an ear canal of the subject;
the display screen coupled for data communications to the image sensor, the display screen displaying images of the ear canal, the display screen positioned on the scanner body in relation to the ear probe so that, when the ear probe is positioned for scanning, both the display screen and the ear probe are visible to an operator operating the scanner; and
the image sensor coupled for data communications to a data processor, with the data processor configured so that it functions by constructing, in dependence upon a sequence of images captured when the scanned ear is illuminated by laser light and tracked positions of the subject's ear probe inferred from reflections of tracking illumination sensed by the tracking illumination sensors, a 3D image of the interior of the ear canal; and
(2) a custom in-ear orthotic modeled from the constructed 3D image of the interior of the subject's ear canal such that the custom orthotic substantially conforms to the subject's ear canal, the in-ear orthotic comprising:
a surface comprising at least one feature that is customized to the subject's ear canal, wherein the at least one feature provides a sensory indication to the subject's ear canal when the subject's jaw moves past a predetermined limit.

16. The system of claim 15, wherein the at least one feature comprises a protrusion that projects from the surface at a customized angle.

17. The system of claim 16, wherein the protrusion is positioned along the in-ear orthotic such that it aligns with either a first bend or a second bend of the subject's ear canal when the in-ear orthotic is inserted in the subject's ear canal.

18. The system of claim 15, wherein the at least one feature is compressible.

19. The system of claim 15, wherein the predetermined limit corresponds to when the subject is clenching the jaw or grinding teeth.

20. The system of claim 15, further comprising at least one sensor that generates a signal when the jaw moves past the predetermined limit.

21. The system of claim 20, wherein the at least one sensor comprises at least one force sensor that senses force on the ear canal associated with the movement of the jaw.

22. The system of claim 20, wherein the at least one sensor comprises at least one accelerometer that detects acceleration of the subject's jaw.

23. The system of claim 20, wherein the at least one sensor comprises at least one voltage sensor that detects voltage across the subject's muscles or nerves.

24. The system of claim 20, wherein the signal is an auditory signal or a vibration.

25. The system of claim 15, wherein the orthotic further comprises a generally C-shaped sound channel.

26. An in-ear device that is customized to a subject's ear canal to substantially deform the subject's ear canal to relieve one or more symptoms associated with temporomandibular joint disorder.
